(19) United States
(12) Patent Application Publication
(10) Pub. No.: US 2013/0012410 A1
(43) Pub. Date: Jan. 10, 2013

(54) METHODS AND MATERIALS FOR DETECTING COLORECTAL CANCER AND ADENOMA

(75) Inventors: Hongzhi Zou, Middleton, WI (US); David A. Ahlquist, Rochester, MN (US)

(73) Assignee: MAYO FOUNDATION FOR MEDICAL EDUCATION AND RESEARCH, Rochester, MN (US)

(21) Appl. No.: 13/575,831

(22) PCT Filed: Mar. 25, 2011
(86) PCT No.: PCT/US2011/029982

§ 371 Date: Sep. 4, 2012

Related U.S. Application Data
(60) Provisional application No. 61/318,670, filed on Mar. 29, 2010.

(51) Int. Cl. C12Q 1/68; G01N 25/04; G01N 21/64; C40B 30/04

(52) U.S. Cl. 506/9; 435/6.11; 435/6.12; 435/6.14

(57) ABSTRACT

The present invention provides methods and materials related to the detection of colorectal neoplasm-specific markers (e.g., markers associated with colorectal cancer, markers associated with adenoma) in or associated with a subject's stool sample. In particular, the present invention provides methods and materials for identifying mammals (e.g., humans) having a colorectal neoplasm by detecting the presence and level of indicators of colorectal neoplasia such as, for example, long DNA (e.g., quantified by Alu PCR) and the presence and level of tumor-associated gene alterations (e.g., mutations in KRAS, APC, melanoma antigen gene, p53, BRAF, BAT26, PIK3CA) or epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, FOXE1) in DNA from a stool sample obtained from the mammal.

### CROSS-REFERENCE TO RELATED APPLICATION

The present application claims priority to pending U.S. Provisional Patent Application No. 61/318,670, filed Mar. 29, 2010, the contents of which are incorporated by reference in its entirety.

### FIELD OF THE INVENTION

The present invention provides methods and materials related to the detection of colorectal neoplasm-specific markers (e.g., markers associated with colorectal cancer, markers associated with adenoma) in or associated with a subject's stool sample. In particular, the present invention provides methods and materials for identifying mammals (e.g., humans) having a colorectal neoplasm by detecting the presence and level of indicators of colorectal neoplasia such as, for example, long DNA (e.g., quantified by Alu PCR) and the presence and level of tumor-associated gene alterations (e.g., mutations in KRAS, APC, melanoma antigen gene, p53, BRAF, BAT26, PIK3CA) or epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, FOXE1) in DNA from a stool sample obtained from the mammal.

### BACKGROUND OF THE INVENTION

Colorectal cancer (CRC) remains a leading cause of death among the types of cancer (see, e.g., Jemal A, et al., CA Cancer J. Clin. 2007, 57:43-66; herein incorporated by reference in its entirety). Although screening reduces colorectal cancer mortality (see, e.g., Mandel J S, et al., N Engl J. Med. 1993, 328:1365-71; Hardcastle J D, et al., Lancet. 1996, 348:1472-7; Kronborg O, et al., Scand J. Gastroenterol. 2004, 39:846-51; Winawer S J, et al., J Natl Cancer Inst. 1993, 85:1311-8; Singh H, et al., JAMA. 2006, 295:2366-73; each herein incorporated by reference in their entireties), observed reductions have been modest (see, e.g., Singh H, et al., JAMA. 2006; 295, 2366-73; Heresbach D, et al., Eur J Gastroenterol Hepatol. 2006, 18:427-33; each herein incorporated by reference in their entireties) and more than one half of adults in the United States have not received screening (see, e.g., Meissner H I, Cancer Epidemiol Biomarkers Prev. 2006, 15:389-94; herein incorporated by reference in its entirety).

CRC is curable when it is diagnosed while still localized and is largely preventable by the detection and removal of advanced adenomas. Because advanced adenomas and CRCs at curable stages are seldom symptomatic, the only effective approach to early detection is to screen members of the population at average risk. As an emerging non-invasive approach, stool-based DNA testing represents an attractive option for CRC screening due to its ease of administration and low cost relative to standard-of-care invasive screening procedures such as colonoscopies and sigmoidoscopy. However, current stool DNA tests are endorsed for screening CRC only, and are not endorsed for screening colorectal adenomas (Levin et al. (2008) CA Cancer J. Clin. 58:130-160; herein incorporated by reference in its entirety). Considering that advanced adenoma is the precursor of CRC, and about eight times more prevalent than CRC in humans aged 50 years and older, more accurate, user-friendly, and widely distributable tools are needed to improve colorectal adenoma screening effectiveness, acceptability, and access.

### SUMMARY

The present invention provides methods and materials related to the detection of colorectal neoplasm-specific markers (e.g., markers associated with colorectal cancer, markers associated with adenoma) in or associated with a subject's stool sample. In particular, the present invention provides methods and materials for identifying mammals (e.g., humans) having a colorectal neoplasm by detecting the presence and level of indicators of colorectal neoplasia such as, for example, long DNA (e.g., quantified by Alu PCR) and the presence and level of tumor-associated gene alterations (e.g., mutations in KRAS, APC, melanoma antigen gene, p53, BRAF, BAT26, PIK3CA) or epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, FOXE1) in DNA from a stool sample obtained from the mammal.

Effective and highly sensitive assays for detecting the presence of colorectal neoplasms (e.g., cancer, adenoma (e.g., advanced adenoma)) are urgently needed in clinical settings, as such assays facilitate diagnosis and clinical intervention at an early stage, thereby leading to much improved rates of recovery and lowering of morbidity and mortality in comparison to diagnostic methods that detect later-stage colorectal cancers. During the course of developing some embodiments of the present invention, multimarker panel assay systems were developed that resulted in higher levels of sensitivity and specificity for detection of colorectal cancer and advanced adenoma than single marker assay systems. In particular, such assay systems included multiple indicators of colorectal neoplasms, such as detecting and characterizing mutation score, mutation frequency, or mutation level in at least two biomarkers (e.g., single point mutations or multiple mutations in mutation cluster region(s) of KRAS, APC, melanoma antigen gene, p53, BRAF, BAT26, PIK3CA); detecting and characterizing methylation score, methylation frequency, or methylation level of one or more CpG island or CpG shore biomarkers (e.g., bmp-3, bmp-4, SFRP2, vimentin, septin9, ALX4, EYA4, TFPI2, NDRG4, FOXE1); and detecting and characterizing the level of long DNA. Additionally, analytical sensitivity was improved by incorporating methods for specimen collection that facilitated DNA integrity in the stool sample, for example, by utilizing stabilization buffer with DNase inhibiting agents such as, for example, chelating agents. Therefore, during experiments conducted in the course of developing some embodiments of the present invention, colorectal cancers were detected at a sensitivity of 91% and advanced adenomas were detected at a sensitivity of 78% when specificity was set at 85%.

Accordingly, in certain embodiments, the present invention provides methods for detecting the presence of a colorectal neoplasm in a mammal. In some embodiments, the methods involve obtaining a stool sample from a mammal, extracting DNA from the stool sample such that the integrity of the DNA is substantially similar to the integrity of the DNA in unexcreted stool from the mammal, and detecting the level of multiple indicators of colorectal neoplasm. The methods of the present invention are not limited to particular indicators of colorectal neoplasm.

In some embodiments, indicators of colorectal neoplasm include, for example, mutated nucleic acids. The methods are not limited to particular mutated nucleic acids for detecting the presence of a colorectal neoplasm in a mammal. In some embodiments, the mutation is a single point mutation in a biomarker of interest. In some embodiments, more than one mutation is present in a biomarker of interest. Mutations may be single base pair deletions, substitutions, or additions; or deletions, substitions, additions, rearrangements (e.g., inversions, transversions) of more than one base pair. Methods of the present invention are not limited by particular biomarkers for detecting mutated nucleic acid. Biomarkers include but are not limited to KRAS, APC, melanoma antigen gene, p53, BRAF, BAT26, and PIK3CA and regions associated with such biomarkers. Mutations in one, two, three, four, or four or more nucleic acid polymers may be detected.

Detection of the presence (e.g., level, frequency, score) of single point mutations is not limited by the technique used for such detection. In some embodiments, techniques used for detection of single point mutations include but are not limited to allele-specific PCR, mutant-enriched PCR, digital protein truncation test, direct sequencing, molecular beacons, and BEAMing. In some embodiments, a region (e.g., a mutation cluster region) is surveyed for level of mutations (e.g., mutation score, mutation frequency) (e.g., presence of multiple mutations), without limitation to the technique used to determine the level of mutation. Techniques used to assess mutation levels in, for example, mutation cluster regions include but are not limited to melt curve analysis, temperature gradient gel electrophoresis, and digital melt curve assay. In some preferred embodiments, digital melt curve assay is used.

In some embodiments, indicators of colorectal neoplasm include, for example, epigenic alterations. Epigenetic alterations include but are not limited to DNA methylation (e.g., CpG methylation). In some embodiments, the level (e.g., frequency, score) of methylation (e.g., hypermethylation relative to a control, hypomethylation relative to a control) is determined without limitation to the technique used for such determining. Methods of the present invention are not limited to particular epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, and FOXE1). In some embodiments, methylation of a CpG island is assessed. In some embodiments, methylation of a CpG island shore is assessed.

Techniques used to assess DNA methylation levels include but are not limited to methylation-specific PCR, quantitative methylation-specific PCR, Restriction Landmark Genomic Scanning for Methylation (RLGS-M), comprehensive high-throughput relative methylation (CHARM) analysis (see, e.g., Irizarry et al. (2009) Nature Gen. 178-186; herein incorporated by reference in its entirety), CpG island microarray, methylated DNA immunopreciptiation, methylation-sensitive DNA restriction enzyme analysis, and bisulfite genomic sequencing PCR, methylation-specific PCR, quantitative methylation-specific PCR, methylation-sensitive DNA restriction enzyme analysis, quantitative bisulfite pyrosequencing, and bisulfite genomic sequencing PCR.

In some embodiments, an indicator of colorectal neoplasm includes the presence (e.g., level, concentration, abundance) of long DNA (e.g., of mammalian origin). While the present invention is not limited to any particular mechanism, and an understanding of the mechanism is not necessary to practice the present invention, it is contemplated that long DNA (e.g., DNA greater 100 base pairs in length, greater than 150 base pairs in length, greater than 200 base pairs in length, greater than 250 base pairs in length, greater than 300 base pairs in length, greater than 500 base pairs in length, greater than 750 base pairs in length, greater than 1000 base pairs in length) is present in stool samples when non-apoptotic cells are exfoliated, wherein such non-apoptotic cells may arise during the development of neoplasias (e.g., adenomas, tumors). The present invention is not limited by methods used to detect long DNA. In some embodiments, A/u_PCR is used to detect long DNA.

The methods are not limited to a particular type of mammal. In some embodiments, the mammal is a human. The methods are not limited to a particular type or stage of colorectal neoplasm. In some embodiments, the colorectal neoplasm is premalignant. In some embodiments, the colorectal neoplasm is malignant. In some embodiments, the colorectal neoplasm is colorectal cancer without regard to stage of the cancer (e.g., stage I, II, III, or IV). In some embodiments, the colorectal neoplasm is adenoma, without regard to the size of the adenoma (e.g., greater than 3 cm; less than or equal to 3 cm; greater than 1 cm; less than or equal to 1 cm). In some embodiments, the adenoma is considered to be an advanced adenoma.

In some embodiments wherein a colorectal neoplasm is detected, additional techniques are performed to characterize the colorectal neoplasm (e.g., to characterize the colorectal neoplasm as malignant or premalignant) (e.g., to characterize the colorectal neoplasm within a particular stage of colorectal cancer).

In some embodiments, methods, kits, and systems of the present invention find use in detecting the presence of colorectal neoplasias. In some embodiments, methods, kits, and systems of the present invention find use in detecting the presence of neoplasias at non-colorectal locations. For example, neoplasms may be located in a mammal's small intestine, gall bladder, bile duct, pancreas, liver, stomach, esophagus, lung, or naso-oro-pharyngeal airway.

In some embodiments, methods, kits, and systems of the present invention are further combined with methods, kits, or systems to detect additional indicators of colorectal neoplasia (e.g., fecal occult blood) (e.g., as detected by assays selected from the group consisting of fecal immunochemical tests (e.g., HemeSelect), fecal porphyrin tests (e.g., Hemoquant), and stool guaiac tests (e.g., Hemoccult, Instacult).

In certain embodiments, the present invention provides kits for detecting the presence of a colorectal neoplasm in a mammal. In some embodiments, such kits include reagents useful, sufficient, or necessary for detecting and/or characterizing one or more indicators specific for a colorectal neoplasm. In some embodiments, the kits contain the reagents necessary to perform real-time Alu PCR. In some embodiments, the kits contain the reagents necessary to perform digital melt curve analysis. In some embodiments, the kits contain the reagents necessary to perform quantitative methylation-specific PCR. In some embodiments, the kits contain the ingredients and reagents necessary to obtain and store a stool sample from a subject.

In certain embodiments, the present invention provides methods for monitoring a treatment of colorectal cancer. For example, in some embodiments, the methods may be performed immediately before, during and/or after a treatment to monitor treatment success. In some embodiments, the methods are performed at intervals on disease-free patients to ensure or monitor treatment success.

In certain embodiments, the present invention provides methods for obtaining a subject's risk profile for developing colorectal cancer. In some embodiments, such methods involve obtaining a stool sample from a subject (e.g., a human at risk for developing colorectal cancer; a human undergoing a routine physical examination), detecting the presence or absence of one or more indicators of colorectal neoplasia (e.g., detecting the presence, absence, or level of markers specific for a colorectal neoplasm in or associated with the stool sample (e.g., mutation level, score or frequency; methylation level, score or frequency; long DNA level)) in the stool sample, and generating a risk profile for developing colorectal cancer based upon the detected presence, absence, or level of the indicators of colorectal neoplasia. For example, in some embodiments, a generated risk profile will change depending upon the level, score, or frequency of specific indicators of colorectal neoplasia. The present invention is not limited to a particular manner of generating the risk profile. In some embodiments, a processor (e.g., computer) is used to generate such a risk profile. In some embodiments, the processor uses an algorithm (e.g., software) specific for interpreting the presence, absence or level of indicators of colorectal neoplasia as determined with the methods of the present invention. In some embodiments, the presence, absence, or level of specific indicators of colorectal neoplasia as determined with the methods of the present invention are inputed into such an algorithm, and the risk profile is reported based upon a comparison of such input with established norms (e.g., established norm for pre-cancerous condition, established norm for various risk levels for developing colorectal cancer, established norm for subjects diagnosed with various stages of colorectal cancer). In some embodiments, the risk profile indicates a subject's risk for developing colorectal cancer or a subject's risk for re-developing colorectal cancer. In some embodiments, the risk profile indicates a subject to be, for example, a very low, a low, a moderate, a high, and a very high chance of developing or re-developing colorectal cancer. In some embodiments, a health care provider (e.g., an oncologist) will use such a risk profile in determining a course of treatment or intervention (e.g., colonoscopy, watchful waiting, referral to an oncologist, referral to a surgeon, etc.).

In certain embodiments, the present invention provides methods for detecting colorectal neoplasia in a subject comprising: obtaining DNA from an excreted stool sample of the subject, wherein the DNA is substantially intact relative to DNA obtained from unexcreted stool from the subject; and determining the level of indicators of colorectal neoplasia in the DNA from the excreted stool sample. The methods are not limited to particular indicators of colorectal neoplasia. Examples of indicators of colorectal neoplasia include but are not limited to nucleic acid polymer methylation (e.g., hypermethylation, hypomethylation), one or more mutated nucleic acid polymers, or long DNA.

In some embodiments, the nucleic acid polymer with altered methylation (e.g., hypermethylated, hypomethylated) comprises a CpG island or CpG shore. In some embodiments, the CpG island or CpG shore is present in a coding region or a regulatory region of a gene such as bmp-3, bmp-4, SFRP2, vimentin, septin9, ALX4, EYA4, TFPI2, NDRG4, or FOXE1. In some embodiments, determining of the level of methylation of a nucleic acid polymer comprises determining the methylation score of the CpG island or CpG shore. In some embodiments, determining the level of methylation of a nucleic acid polymer comprises determining the methylation frequency of the CpG island or CpG shore. In some embodiments, determining the level of methylation of a nucleic acid polymer is achieved by a technique such as methylation-specific PCR, quantitative methylation-specific PCR, methylation-sensitive DNA restriction enzyme analysis, quantitative bisulfite pyrosequencing, or bisulfite genomic sequencing PCR.

In some embodiments, the mutated nucleic acid polymer comprises a point mutation in a gene such as KRAS, APC, melanoma antigen gene, p53, or PIK3CA. In some embodiments, the mutated nucleic acid polymer comprises more than one mutation in a gene such as KRAS, APC, melanoma antigen gene, p53, and PIK3CA. In some embodiments, the point mutation is detected by a technique such as allele-specific PCR, mutant-enriched PCR, digital protein truncation test, direct sequencing, molecular beacons, or BEAMing. In some embodiments, determining the level of more than one mutations involves, for example, determining the mutation score of the nucleic acid polymer. In some embodiments, determining the level of more than one mutations comprises ascertaining the mutation frequency of the nucleic acid polymer. In some embodiments, the mutation score or mutation frequency is detected by a technique such as melt curve analysis, temperature gradient gel electrophoresis, or digital melt curve assay. In some embodiments the level of long DNA is detected using Alu PCR assay.

In some embodiments, methods of the present invention further comprise generating a risk profile using the results of steps described supra. In some embodiments, the colorectal neoplasm is premalignant. In some embodiments, the colorectal neoplasm is malignant. In some embodiments, the indicators of colorectal neoplasia comprise KRAS mutation level, APC mutation score or mutation level, BMP3 methylation level, and long DNA concentration. In some embodiments, the method permits detection of colorectal cancer in said subject with a sensitivity of at least 85% at a specificity of at least 85%. In some embodiments, the method permits detection of colorectal cancer in said subject with a sensitivity of at least 80% at a specificity of at least 90%. In some embodiments, the method permits detection of colorectal adenoma in said subject with a sensitivity of at least 75% at a specificity of at least 85%. In some embodiments, the method permits detection of colorectal adenoma in said subject with a sensitivity of at least 60% at a specificity of at least 90%. In some embodiments, the long DNA comprises DNA greater than 200 base pairs in length.

In certain embodiments, the present invention provides a kit for detecting the presence of a colorectal neoplasm in a mammal, the kit comprising reagents useful, sufficient, or necessary for detecting and/or characterizing indicators of colorectal neoplasia in DNA from a stool sample, the indicators of types such as a nucleic acid polymer with altered methylation, one or more mutated nucleic acid polymers, and/or long DNA. In some embodiments, the indicators of colorectal neoplasma are indicators such as presence or frequency of mutations in coding or regulatory regions of KRAS, APC, melanoma antigen gene, p53, and/or PIK3CA; level or frequency of CpG methylation in coding or regulatory regions of bmp-3, bmp-4, SFRP2, vimentin, septin9, ALX4, EYA4, TFPI2, NDRG4, FOXE1; and presence or concentration of long DNA.

Additional embodiments will be apparent to persons skilled in the relevant art based on the teachings contained herein.

### BRIEF DESCRIPTION OF THE DRAWINGS

FIG. 1 shows a design of a digital melt curve (DMC) assay for deeply scanning an APC gene mutator cluster region (MCR). A total of 26 primer pairs, named as primers A to Z (horizontal bars) were designed to scan the two mutation-dense regions between codons 1286-1379 and between codons 1387-1499, and one less dense region between codons 1537-1554. For optimal mutation detection, amplicons were shorter than 400 bp. After stepwise selection, primer pairs C and N (horizontal black bars with arrow ends) were used as APC assays 1 and 2 with 201 stool samples. Vertical bars indicate the reported common mutation sites in APC MCR, and their lengths represent mutation density. The numbers below the top scale indicate the locations of amino acids/codons in APC protein/gene.

FIG. 2 shows detection of APC mutations in stools from patients and normal individuals with digital melt curve assays. Representative shifted melt curves demonstrate that APC mutations were detected more frequently in stools from patients with colorectal cancer or advanced adenomas than from normal individuals. Background mutations could be detected in stools from normal individuals. Lower lines (solid arrows) represent positive wells on a 96-well plate with formation of mutant/wild-type heteroduplex, and upper lines (dashed arrows) represent normal wells with wild-type homoduplex. Heteroduplex melts at slightly lower temperature, causing the shift in the melt curve.

FIG. 3A shows the mutation score of the logistically combined DMC APC assay with assays 1 and 2 in stools from patients with colorectal cancers or advanced adenomas and from normal individuals (see, e.g., Example 1). The combined mutation score was displayed in log scale. Each circle represents one stool sample. FIG. 3B shows methylation of BMP3 as assessed using quantitative methylation-specific PCR (see, e.g., Example 1). Median copies of methylated BMP3 were 200 (range, 0-110933), 108 (0-3195), and 0 (0-1800) copies/g stool for CRC patients, adenoma patients, and normal controls, respectively. Each circle represents one stool sample.

FIG. 4 shows receiver operating curves (ROCs) for APC mutation scores. A, ROCs in stools from CRC patients versus from normal individuals. APC assays 1 and 2 and their logistic combination are displayed together in one graph. B, ROCs in stools from adenoma patients versus normal individuals. APC assays 1 and 2 and their logistic combination are displayed together in one graph.

FIG. 5 shows the mutation scores of digital melt curve APC assays 1 and 2 in stools from patients with CRCs or advanced adenomas and from normal individuals. APC assays 1 and 2 are displayed separately. Each circle represents one stool sample.

### DEFINITIONS

To facilitate an understanding of the present invention, a number of terms and phrases are defined below.

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J. Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 100 or more by in length. In some embodiments, CpG islands show altered methylation patterns relative to controls (e.g., altered methylation in cancer subjects relative to subjects without cancer; tissue-specific altered methylation patterns; altered methylation in stool from subjects with colorectal neoplasia (e.g., colorectal cancer, colorectal adenoma) relative to subjects without colorectal neoplasia). In some embodiments, altered methylation involves hypermethylation. In some embodiments, altered methylation involves hypomethylation.

As used herein, the term “CpG shore” or “CpG island shore” refers to a genomic region external to a CpG island that is or that has potential to have altered methylation patterns (see, e.g., Irizarry et al. (2009) Nat. Genetics 41:178-186; herein incorporated by reference in its entirety). CpG island shores may show altered methylation patterns relative to controls (e.g., altered methylation in cancer subjects relative to subjects without cancer; tissue-specific altered methylation patterns; altered methylation in stool from subjects with colorectal neoplasia (e.g., colorectal cancer, colorectal adenoma) relative to subjects without colorectal neoplasia). In some embodiments, altered methylation involves hypermethylation. In some embodiments, altered methylation involves hypomethylation. CpG island shores may be located in various regions relative to CpG islands (see, e.g., Irizarry et al. (2009) Nat. Genetics 41; 178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

As used herein, the term “colorectal cancer” is meant to include the well-accepted medical definition that defines colorectal cancer as a medical condition characterized by cancer of cells of the intestinal tract below the small intestine (e.g., the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum).

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized colorectal cancer cells” is meant to refer to colorectal cancer cells which have metastasized; colorectal cancer cells localized in a part of the body other than the duodenum, small intestine (jejunum and ileum), large intestine (colon), including the cecum, ascending colon, transverse colon, descending colon, and sigmoid colon, and rectum.

As used herein, “an individual is suspected of being susceptible to metastasized colorectal cancer” is meant to refer to an individual who is at an above-average risk of developing metastasized colorectal cancer. Examples of individuals at a particular risk of developing metastasized colorectal cancer are those whose family medical history indicates above average incidence of colorectal cancer among family members and/or those who have already developed colorectal cancer and have been effectively treated who therefore face a risk of relapse and recurrence. Other factors which may contribute to an above-average risk of developing metastasized colorectal cancer which would thereby lead to the classification of an individual as being suspected of being susceptible to metastasized colorectal cancer may be based upon an individual's specific genetic, medical and/or behavioral background and characteristics.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells. The term “colorectal neoplasm-specific marker” refers to any biological material that can be used to indicate the presence of a colorectal neoplasm (e.g., a premalignant colorectal neoplasm; a malignant colorectal neoplasm). Examples of colorectal neoplasm-specific markers include, but are not limited to, mutated or hypermethlated markers (e.g., bmp-3, bmp-4, SFRP2, vimentin, septin9, ALX4, EYA4, TFPI2, NDRG4, FOXE1, long DNA, K-ras, APC, melanoma antigen gene, p53, and PIK3CA) and long DNA.

As used herein, the term “adenoma” refers to a benign tumor of glandular origin. Although these growths are benign, over time they may progress to become malignant. As used herein the term “colorectal adenoma” refers to a benign colorectal tumor in which the cells form recognizable glandular structures or in which the cells are clearly derived from glandular epithelium.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specfic PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

### DETAILED DESCRIPTION OF THE INVENTION

Stool DNA testing is an attractive option for screening colorectal neoplasia; however, prior to the development of some embodiments of the present invention, stool DNA tests held limited ability to detect colorectal adenomas with sufficient sensitivity and specificity. In the course of developing some embodiments of the present invention it was found that a multi-marker quantitative stool DNA testing could detect both CRC and advanced adenoma at high sensitivities by incorporating a stabilization buffer in sample preparation, sensitive assay platform, and markers with broad coverage. For example, when specificity was set at 85%, the new quantitative stool DNA testing with fecal long DNA, KRAS and APC mutations, and BMP3 methylation could detect 91% colorectal cancers and 78% advanced adenomas. In addition it was found that optimal performance was facilitated by inhibiting human DNA degradation in stool samples or in DNA extracted from stool samples. Inhibition of DNases prevalent in stool is important to assay system performance; for example, stool samples collected without preservative buffer and transported at room temperature may result in degraded of significant human DNA in the sample prior to DNA extraction. For example, it was found that fecal human DNA levels fell as much as 75% after storage for one day at room temperature (see, e.g., Zou et al. (2006) Cancer Epidemiol. Biomarkers Prev. 15:1115-1119; herein incorporated by reference in its entirety).

Accordingly, the present invention provides methods and materials related to the detection of colorectal neoplasm-specific markers (e.g., markers associated with colorectal cancer, markers associated with adenoma) in or associated with a subject's stool sample. In particular, the present invention provides methods and materials for identifying mammals (e.g., humans) having a colorectal neoplasm by detecting the presence and level of indicators of colorectal neoplasia such as, for example, long DNA (e.g., quantified by Alu PCR) and the presence and level of tumor-associated gene alterations (e.g., mutations in KRAS, APC, melanoma antigen gene, p53, BRAF, BAT26, PIK3CA) or epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, FOXE1) in DNA from a stool sample obtained from the mammal.

While the present invention exemplifies several markers specific for detecting colorectal cancer, any marker that is correlated with the presence or absence of colorectal cancer may be used. A marker, as used herein, includes, for example, nucleic acid(s) whose production or mutation or lack of production is characteristic of a colorectal neoplasm. Depending on the particular set of markers employed in a given analysis, the statistical analysis will vary. For example, where a particular combination of markers is highly specific for colorectal cancer, the statistical significance of a positive result will be high. It may be, however, that such specificity is achieved at the cost of sensitivity (e.g., a negative result may occur even in the presence of colorectal cancer or colorectal adenoma). By the same token, a different combination may be very sensitive (e.g., few false negatives, but has a lower specificity).

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

The methods of the present invention are not limited to particular indicators of colorectal neoplasm.

In some embodiments, indicators of colorectal neoplasm include, for example, mutated nucleic acids. The methods are not limited to particular mutated nucleic acids for detecting the presence of a colorectal neoplasm in a mammal. In some embodiments, the mutation is a single point mutation in a biomarker of interest. In some embodiments, more than one mutation is present in a biomarker of interest. Mutations may be single base pair deletions, substitutions, or additions; or deletions, substitions, additions, rearrangements (e.g., inversions, transversions) of more than one base pair. Methods of the present invention are not limited by particular biomarkers for detecting mutated nucleic acid. Biomarkers include but are not limited to KRAS, APC, melanoma antigen gene, p53, and PIK3CA.

In some embodiments, indicators of colorectal neoplasm include, for example, epigenic alterations. Epigenetic alterations include but are not limited to DNA methylation (e.g., CpG methylation). In some embodiments, the level (e.g., frequency, score) of methylation (e.g., hypermethylation relative to a control, hypomethylation relative to a control) is determined without limitation to the technique used for such determining. Methods of the present invention are not limited to particular epigenetic alterations (e.g., DNA methylation) (e.g., CpG methylation) (e.g., CpG methylation in coding or regulatory regions of bmp-3, bmp-4, SFRP2, vimentin, septin9, ALX4, EYA4, TFPI2, NDRG4, and FOXE1). Altered methylation may occur in, for example, CpG islands; CpG island shores; or regions other than CpG islands or CpG island shores.

In some embodiments, indicators of colorectal neoplasm include, for example, nucleic acid that reflects microsatellite instabilty. Nucleic acid that reflects microsatellite instability can be used to indicate the presence of colorectal cancer. Briefly, nucleic acid that reflects microsatellite instability can be identified as described elsewhere (see, e.g., Samowitz et al., Am. J. Path., 154:1637-1641 (1999); Hoang et al., Cancer Res., 57:300-303 (1997); each herein incorporated by reference in its entirety). Examples of nucleic acid that can reflect microsatellite instability indicative of a colorectal neoplasm includes, without limitation, the gene for BAT-26 and BRAF.

In some embodiments, an indicator of colorectal neoplasm is the presence (e.g., level, concentration, abundance) of long DNA (e.g., of mammalian origin). While the present invention is not limited to any particular mechanism, and an understanding of the mechanism is not necessary to practice the present invention, it is contemplated that long DNA (e.g., DNA greater 100 base pairs in length, greater than 150 base pairs in length, greater than 200 base pairs in length, greater than 250 base pairs in length, greater than 300 base pairs in length, greater than 500 base pairs in length, greater than 750 base pairs in length, greater than 1000 base pairs in length) is present in stool samples when non-apoptotic cells are exfoliated, wherein such non-apoptotic cells may arise during the development of neoplasia (e.g., adenomas, tumors).

The present invention is not limited to a particular method for detecting and/or quantifying long DNA within a subject's stool sample. In some embodiments, real-time Alu PCR is used for detecting and/or quantifying long DNA within a subject's stool sample. Real-time Alu PCR is a sensitive method for detecting non-apoptotic human DNA in stool as it targets abundant Alu repeats in human genome (see, e.g., Zou H, et al. Cancer Epidemiol Biomarkers Prev 2006, 15:1115-1119; herein incorporated by reference in its entirety). Alu sequences embody the largest family of middle repetitive DNA sequences in human genome (see, e.g., Kariya Y, et al., Gene 1987, 53:1-10; herein incorporated by reference in its entirety). An estimated half million Alu copies are present per haploid human genome (see, e.g., Kariya Y, et al., Gene 1987, 53:1-10; herein incorporated by reference in its entirety). Accordingly, as Alu sequences are so abundantly distributed throughout the genome and specific to the genomes primates, real-time Alu PCR amplifies DNA sequences longer than 200 bp within these 300-bp repeats (see, e.g., Kariya Y, et al., Gene 1987, 53:1-10; herein incorporated by reference in its entirety) thereby providing a genome-wide approach to quantify human long DNA in stool (see, e.g., Zou H, et al. Cancer Epidemiol Biomarkers Prev 2006, 15:1115-1119; herein incorporated by reference in its entirety).

In certain embodiments, methods, kits, and systems of the present invention involve determination of methylation state of a locus of interest (e.g., in human DNA) (e.g., in human DNA extracted from a stool sample). Any appropriate method can be used to determine whether a particular DNA is hypermethylated or hypomethylated. Standard PCR techniques, for example, can be used to determine which residues are methylated, since unmethylated cytosines converted to uracil are replaced by thymidine residues during PCR. PCR reactions can contain, for example, 100 μL of captured DNA that either has or has not been treated with sodium bisulfite, 1×PCR buffer, 0.2 mM dNTPs, 0.5 μM sequence specific primers (e.g., primers flanking a CpG island or CpG shore within the captured DNA), and 5 units DNA polymerase (e.g., Amplitaq DNA polymerase from PE Applied Biosystems, Norwalk, Conn.) in a total volume of 50 μl. A typical PCR protocol can include, for example, an initial denaturation step at 94° C. for 5 min, 40 amplification cycles consisting of 1 minute at 94° C., 1 minute at 60° C., and 1 minute at 72° C., and a final extension step at 72° C. for 5 minutes.

To analyze which residues within a captured DNA are methylated, the sequences of PCR products corresponding to samples treated with and without sodium bisulfite can be compared. The sequence from the untreated DNA will reveal the positions of all cytosine residues within the PCR product. Cytosines that were unmethylated will be converted to thymidine residues in the sequence of the bisulfite-treated DNA, while residues that were methylated will be unaffected by bisulfite treatment.

Purified nucleic acid fragments from a stool sample or samples can be analyzed to determine the presence or absence of one or more somatic mutations. Mutations can be single base changes, short insertion/deletions, or combinations thereof. Methods of analysis can include conventional Sanger based sequencing, pyrosequencing, next generation sequencing, single molecule sequencing, and sequencing by synthesis. In some cases, mutational status can be determined by digital PCR followed by high resolution melting curve analysis (digital melt curve, or DMC). In other cases, allele-specific primers or probes in conjunction with amplification methods can be used to detect specific mutations in stool DNA. The mutational signature can comprise not only the event of a base or sequence change in a specific gene, but also the location of the change within the gene, whether it is coding, non-coding, synonymous or non-synonymous, a transversion or transition, and the dinucleotide sequence upstream and downstream from the alteration.

In some embodiments, methods of the present invention involve the determination (e.g., assessment, ascertaining, quantitation) of mutation level of an indicator of colorectal neoplasm (e.g., the mutation level of a mutation cluster region in the coding or regulatory region of a gene locus) in a sample (e.g., a DNA sample extracted from stool). A skilled artisan understands that an increased, decreased, informative, or otherwise distinguishably different mutation level is articulated with respect to a reference (e.g., a reference level, a control level, a threshold level, or the like). For example, the term “increased mutation level” as used herein with respect to the level of a locus (e.g., KRAS, APC) is any mutation level (e.g., mutation frequency, mutation score) that is above a median mutation level (e.g., mutation frequency, mutation score) in a stool sample from a random population of mammals {e.g., a random population of 10, 20, 30, 40, 50, 100, or 500 mammals) that do not have a colorectal neoplasm (e.g., colorectal cancer, colorectal adenoma). An elevated (e.g., increased) mutation level can be any level provided that the level is greater than a corresponding reference level. For example, an elevated mutation level (e.g., mutation score, mutation frequency) of a locus of interest (e.g., KRAS, APC) can be 0.5, 1, 2, 3, 4, 5, 6, 7, 8, 9, 10, or more fold greater than the reference level of the corresponding locus in a normal sample. It is noted that a reference level can be any amount. For example, a reference mutation level for locus can be zero (e.g., no mutations occurring). In some cases, an increased mutation level of a locus can be any detectable level of mutation in DNA extracted from a stool sample.

Similarly, in some embodiments, methods of the present invention involve the determination (e.g., assessment, ascertaining, quantitation) of methylation level of an indicator of colorectal neoplasm (e.g., the mutation level of a CpG island or CpG shore in the coding or regulatory region of a gene locus) in a sample (e.g., a DNA sample extracted from stool). A skilled artisan understands that an increased, decreased, informative, or otherwise distinguishably different methylation level is articulated with respect to a reference (e.g., a reference level, a control level, a threshold level, or the like). For example, the term “elevated methylation” as used herein with respect to the methylation status (e.g., CpG DNA methylation) of a gene locus (e.g., BMP3, ALX, vimentin) is any methylation level that is above a median methylation level in a stool sample from a random population of mammals (e.g., a random population of 10, 20, 30, 40, 50, 100, or 500 mammals) that do not have a colorectal neoplasm (e.g., colorectal cancer, colorectal adenoma). Elevated levels of methylation can be any level provided that the level is greater than a corresponding reference level. For example, an elevated methylation level of a locus of interest (e.g., BMP3, ALX, vimentin) methylation can be 0.5, 1, 2, 3, 4, 5, 6, 7, 8, 9, 10, or more fold greater than the reference level methylation observed in a normal stool sample. It is noted that a reference level can be any amount. The term “elevated methylation score” as used herein with respect to detected methylation events in a matrix panel of particular nucleic acid markers is any methylation score that is above a median methylation score in a stool sample from a random population of mammals (e.g., a random population of 10, 20, 30, 40, 50, 100, or 500 mammals) that do not have a colorectal neoplasm (e.g., colorectal cancer, colorectal adenoma). An elevated methylation score in a matrix panel of particular nucleic acid markers can be any score provided that the score is greater than a corresponding reference score. For example, an elevated score of methylation in a locus of interest (e.g., BMP3, ALX, vimentin) can be 0.5, 1, 2, 3, 4, 5, 6, 7, 8, 9, 10, or more fold greater than the reference methylation score observed in a normal stool sample. It is noted that a reference score can be any amount.

In some cases, a matrix marker panel can be used to identify mammals having a colorectal neoplasia (e.g., a colorectal cancer, colorectal adenoma). In some cases, such panel also can identify the location of the colorectal neoplasia. Such a panel can include nucleic acid markers and combinations thereof and can provide information about a mutated marker gene, the mutated region of the marker gene, and/or type of mutation. For example, data can be analyzed using a statistical model to predict tumor site (e.g., anatomical location or tissue of origin) based on inputs from sequencing data (such as by specific nucleic acid or combination of nucleic acids mutated, specific mutational location on a nucleic acid, and nature of mutation (e.g. insertion, deletion, transition, or transversion) or by any combination thereof) and/or data from polypeptide or other types of markers. For example, a Site of Tumor Estimate (SITE) model can be used to predict tumor site using a matrix panel of markers that are present to variable extent across tumors.

In some cases, data can be analyzed using quantified markers to create a logistic model, which can have both high sensitivity and high specificity. For example, a logistic model can also incorporate population variables like gender and age to adjust cut-off levels for test positivity and thereby optimize assay performance in a screening setting. In some cases, a Quantitative Logistic to Enhance Accurate Detection (Q-LEAD) Model can be used with any marker class or combination of markers as long as they can be quantified.

The methods are not limited to a particular type of mammal. In some embodiments, the mammal is a human. In some embodiments, the colorectal neoplasm is premalignant. In some embodiments, the colorectal neoplasm is malignant. In some embodiments, the colorectal neoplasm is colorectal cancer without regard to stage of the cancer (e.g., stage I, II, III, or IV). In some embodiments, the colorectal neoplasm is adenoma, without regard to the size of the adenoma (e.g., greater than 3 cm; less than or equal to 3 cm; greater than 1 cm; less than or equal to 1 cm). In some embodiments, the adenoma is considered to be an advanced adenoma.

The present invention also provides methods and materials to assist medical or research professionals in determining whether or not a mammal has a colorectal neoplasm (e.g., colorectal cancer, colorectal adenoma). Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the ratio of particular markers in a stool sample, and (2) communicating information about the ratio to that professional, for example. In some cases, a professional can be assisted by (1) determining the level of long DNA, the methylation status of genes such as BMP3, and the mutation score of genes such as APC and K-ras, and (2) communicating information about the level of DNA, the methylation status of particular genes, and the mutation score of particular genes to the professional. In some cases, a professional can be assisted by (1) detecting mutations in cancer-related genes such as K-ras, p53, APC, p16, EGFR, CTNNB1, and SMAD4, in some embodiments in combination with determining the level of long DNA, as a multi-marker panel, and (2) communicating information regarding the markers to the professional.

After the level (score, frequency) of particular markers in a stool sample is reported, a medical professional can take one or more actions that can affect patient care. For example, a medical professional can record the results in a patient's medical record. In some cases, a medical professional can record a diagnosis of a colorectal neoplasia, or otherwise transform the patient's medical record, to reflect the patient's medical condition. In some cases, a medical professional can review and evaluate a patient's entire medical record, and assess multiple treatment strategies, for clinical intervention of a patient's condition. In some cases, a medical professional can record a prediction of tumor occurrance with the reported indicators. In some cases, a medical professional can review and evaluate a patient's entire medical record and assess multiple treatment strategies, for clinical intervention of a patient's condition.

A medical professional can initiate or modify treatment of a colorectal neoplasm after receiving information regarding the level (score, frequency) associated with markers in a patient's stool sample. In some cases, a medical professional can compare previous reports and the recently communicated level (score, frequency) of markers, and recommend a change in therapy. In some cases, a medical professional can enroll a patient in a clinical trial for novel therapeutic intervention of colorectal neoplasm. In some cases, a medical professional can elect waiting to begin therapy until the patient's symptoms require clinical intervention.

A medical professional can communicate the assay results to a patient or a patient's family. In some cases, a medical professional can provide a patient and/or a patient's family with information regarding colorectal neoplasia, including treatment options, prognosis, and referrals to specialists, e.g., oncologists and/or radiologists. In some cases, a medical professional can provide a copy of a patient's medical records to communicate assay results to a specialist. A research professional can apply information regarding a subject's assay results to advance colorectal neoplasm research. For example, a researcher can compile data on the assay results, with information regarding the efficacy of a drug for treatment of colorectal neoplasia to identify an effective treatment. In some cases, a research professional can obtain assay results to evaluate a subject's enrollment, or continued participation in a research study or clinical trial. In some cases, a research professional can classify the severity of a subject's condition, based on assay results. In some cases, a research professional can communicate a subject's assay results to a medical professional. In some cases, a research professional can refer a subject to a medical professional for clinical assessment of colorectal neoplasia, and treatment thereof. Any appropriate method can be used to communicate information to another person (e.g., a professional). For example, information can be given directly or indirectly to a professional. For example, a laboratory technician can input the assay results into a computer-based record. In some cases, information is communicated by making a physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

It is noted that a single stool sample can be analyzed for one colorectal neoplasm-specific marker or for multiple colorectal neoplasm-specific markers. In preferred embodiments, a single stool sample is analyzed for multiple colorectal neoplasm-specific markers, for example, using multi-marker assays. In addition, multiple stool samples can be collected for a single mammal and analyzed as described herein. Indeed, U.S. Pat. Nos. 5,670,325, 5,741,650, 5,928,870, 5,952,178, and 6,020,137, each herein incorporated by reference in their entireties, for example, describe various methods that can be used to prepare and analyze stool samples. In some embodiments, a stool sample is split into first and second portions, where the first portion undergoes analysis for long DNA and the second portion undergoes further purification or processing (e.g., sequence-specific capture step(s) (e.g., for isolation of specific markers) (e.g., for isolation of specific markers for analysis of mutation levels, for isolation of specific markers for analysis of methylation levels). In some embodiments, the stool sample undergoes one or more preprocessing steps before being split into portions. In some embodiments, the stool sample is treated, handled, or preserved in a manner that promotes DNA integrity and/or inhibits DNA degradation (e.g., through use of storage buffers with stabilizing agents (e.g., chelating agents, DNase inhibitors) or handling or processing techniques that promote DNA integrity (e.g., immediate processing or storage at low temperature (e.g., −80 degrees C.)).

The present invention is not limited to a particular manner of detecting nucleic acid markers corresponding to colorectal neoplasm from a stool sample. In some embodiments, nucleic acid is amplified. Generally, nucleic acid used as template for amplification is isolated from cells contained in the biological sample according to standard methodologies (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). The nucleic acid may be genomic DNA or fractionated or whole cell RNA. Where RNA is used, it may be desired to convert the RNA to a complementary cDNA. In a preferred embodiment, the RNA is whole cell RNA and is used directly as the template for amplification. Pairs of primers that selectively hybridize to genes corresponding to specific markers are contacted with the isolated nucleic acid under conditions that permit selective hybridization. Once hybridized, the nucleic acid primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In some applications, the detection may be performed by visual means. Alternatively, the detection may involve indirect identification of the product via chemiluminescence, radioactive scintigraphy of incorporated radio label or fluorescent label or even via a system using electrical or thermal impulse signals. Generally, the foregoing process is conducted at least twice on a given sample using at least two different primer pairs specific for two different specific markers. Following detection, in some embodiments, the results seen in a given subject are compared with a statistically significant reference group of subjects diagnosed as not having colorectal cancer.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

In most cases, it will be preferable to synthesize desired oligonucleotides. Suitable primers can be synthesized using commercial synthesizers using methods well known to those of ordinary skill in the art. Where double-stranded primers are desired, synthesis of complementary primers is performed separately and the primers mixed under conditions permitting their hybridization.

Selection of primers is based on a variety of different factors, depending on the method of amplification and the specific marker involved. For example, the choice of primer will determine the specificity of the amplification reaction. The primer needs to be sufficiently long to specifically hybridize to the marker nucleic acid and allow synthesis of amplification products in the presence of the polymerization agent and under appropriate temperature conditions. Shorter primer molecules generally require cooler temperatures to form sufficiently stable hybrid complexes with the marker nucleic acid and may be more susceptible to non-specific hybridization and amplification.

Primer sequences do not need to correspond exactly to the specific marker sequence. Non-complementary nucleotide fragments may be attached to the 5′ end of the primer with the remainder of the primer sequence being complementary to the template. Alternatively, non-complementary bases can be interspersed into the primer, provided that the primer sequence has sufficient complementarily, in particular at the 3′ end, with the template for annealing to occur and allow synthesis of a complementary DNA strand.

In some embodiments, primers may be designed to hybridize to specific regions of the marker nucleic acid sequence. For example, GC rich regions are favored as they form stronger hybridization complexes than AT rich regions. In another example, primers are designed, solely, to hybridize to a pair of exon sequences, with at least one intron in between. This allows for the activity of a marker gene to be detected as opposed to its presence by minimizing background amplification of the genomic sequences and readily distinguishes the target amplification by size. Primers also may be designed to amplify a particular segment of marker nucleic acid that encodes restriction sites. A restriction site in the final amplification product would enable digestion at that particular site by the relevant restriction enzyme to produce two products of a specific size. Any restriction enzyme may be utilized in this aspect. This added refinement to the amplification process may be necessary when amplifying a marker nucleic acid sequence with close sequence similarity to other nucleic acids. Alternatively, it may be used as an added confirmation of the specificity of the amplification product.

A number of template dependent processes are available to amplify the marker sequences present in a given template sample. One of the best known amplification methods is the polymerase chain reaction (PCR) (see, e.g., U.S. Pat. Nos. 4,683,195, 4,683,202, 4,800,159, and Innis et al., PCR Protocols, Academic Press, Inc., San Diego, Calif. (1990); each incorporated herein by reference in their entireties). Briefly, in PCR, two primer sequences are prepared which are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. In some embodiments, a reverse transcriptase PCR amplification procedure is performed in order to quantify the amount of mRNA amplified. Methods of reverse transcribing RNA into cDNA are well known (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Alternatively, methods for reverse transcription utilize thermostable DNA polymerases (see, e.g., WO 90/07641; herein incorporated by reference in its entirety).

The present invention is not limited to a particular PCR technique. Examples of PCR include, but are not limited to, standard PCR, allele-specific PCR, assembly PCR, asymmetric PCR, digital PCR, helicase-dependent amplification, Hot-start PCR, intersequence-specfic PCR, inverse PCR, ligation-mediated PCR, methylation-specific PCR, miniprimer PCR, multiplex ligation-dependent probe amplification, nested PCR, overlap-extension PCR, real-time PCR, reverse transcription PCR, solid phase PCR, thermal asymmetric interlaced PCR, and Touchdown PCR.

Another method for amplification is the ligase chain reaction (“LCR”) (see, e.g., U.S. Pat. Nos. 4,883,750 and 5,494,810; herein incorporated by reference in its entirety). In LCR, two complementary probe pairs are prepared, and in the presence of the marker sequence, each pair will bind to opposite complementary strands of the marker such that they abut. In the presence of a ligase, the two probe pairs will link to form a single unit. By temperature cycling, as in PCR, bound ligated units dissociate from the marker and then serve as “target sequences” for ligation of excess probe pairs.

Following amplification, it may be desirable to separate the amplification product from the template and the excess primer for the purpose of determining whether specific amplification occurred. In some embodiments, amplification products are separated by agarose, agarose-acrylamide or polyacrylamide gel electrophoresis using standard methods (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (see, e.g., Freifelder, D. Physical Biochemistry Applications to Biochemistry and Molecular Biology. 2nd ed. Wm. Freeman & Co., New York, N.Y. 1982; incorporated herein by reference in its entirety). In some embodiments, amplification product(s) are detected and/or quantified using mass spectrometry techniques.

Amplification products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In some embodiments, visualization is achieved indirectly. For example, following separation of amplification products, a nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety. In some embodiments, detection is by Southern blotting and hybridization with a labeled probe. The techniques involved in Southern blotting are well known to those of skill in the art and can be found in many standard books on molecular protocols (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Briefly, amplification products are separated by gel electrophoresis. The gel is then contacted with a membrane, such as nitrocellulose, permitting transfer of the nucleic acid and non-covalent binding. Subsequently, the membrane is incubated with a chromophore conjugated probe that is capable of hybridizing with a target amplification product. Detection is by exposure of the membrane to x-ray film or ion-emitting detection devices.

In some embodiments, all the basic essential materials and reagents required for detecting colorectal neoplasia through detecting both the level (presence, absence, score, frequency) of markers (e.g., long DNA, neoplasm associated nucleic acid alterations) in a stool sample obtained from the mammal are assembled together in a kit. Such kits generally comprise, for example, reagents useful, sufficient, or necessary for detecting and/or characterizing one or more markers specific for a colorectal neoplasm (e.g., mutations and/or methylations in bmp-3, bmp-4, SFRP2, vimentin, septin9, ALX4, EYA4, TFPI2, NDRG4, FOXE1, long DNA, BAT-26, K-ras, APC, melanoma antigen gene, p53, BRAF, and PIK3CA). In some embodiments, the kits contain enzymes suitable for amplifying nucleic acids including various polymerases, deoxynucleotides and buffers to provide the necessary reaction mixture for amplification. In some embodiments, the kits contain reagents necessary to perform Real-time Alu PCR. In some embodiments, the kits of the present invention include a means for containing the reagents in close confinement for commercial sale such as, e.g., injection or blow-molded plastic containers into which the desired reagent are retained. Other containers suitable for conducting certain steps of the disclosed methods also may be provided.

In some embodiments, the methods disclosed herein are useful in monitoring the treatment of colorectal neoplasia (e.g., colorectal cancer, colorectal adenoma). For example, in some embodiments, the methods may be performed immediately before, during and/or after a treatment to monitor treatment success. In some embodiments, the methods are performed at intervals on disease free patients to insure treatment success.

The present invention also provides a variety of computer-related embodiments. Specifically, in some embodiments the invention provides computer programming for analyzing and comparing a pattern of colorectal neoplasm-specific marker (e.g., long DNA, mutation level of a colorectal neoplasm-specific gene, methylation level of a colorectal neoplasm-specific gene) detection results in a stool sample obtained from a subject to, for example, a library of such marker patterns known to be indicative of the presence or absence of a colorectal neoplasm, or a particular stage or colorectal neoplasm.

In some embodiments, the present invention provides computer programming for analyzing and comparing a first and a second pattern of colorectal neoplasm-specific marker detection results from a stool sample taken at at least two different time points. In some embodiments, the first pattern may be indicative of a pre-cancerous condition and/or low risk condition for colorectal cancer and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of colorectal neoplasm-specific marker detection results from a stool sample to a library of colorectal neoplasm-specific marker patterns known to be indicative of the presence or absence of a colorectal cancer, wherein the comparing provides, for example, a differential diagnosis between a benign colorectal neoplasm, and an aggressively malignant colorectal neoplasm (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of colorectal neoplasm-specific marker detection results from a stool sample). This information received can be stored at least temporarily in a database, and data analyzed in comparison to a library of marker patterns known to be indicative of the presence or absence of a pre-cancerous condition, or known to be indicative of a stage and/or grade of colorectal cancer and/or colorectal adenoma.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of colorectal neoplasm-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a stool sample obtained from a subject is to be input by a user (e.g., a technician or someone performing the assays)) and transmitted to a remote site to a second computer processor for analysis (e.g., where the pattern of colorectal neoplasm-specific marke) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of a colorectal neoplasm, or monitoring the progression of a pre-cancerous condition or a colorectal neoplasm. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of a colorectal neoplasm, or a relational database (RDB) which can include data input by the user and data output. The computer processors can be processors that are typically found in personal desktop computers (e.g., IBM, Dell, Macintosh), portable computers, mainframes, minicomputers, or other computing devices.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing colorectal neoplasm (e.g., colorectal cancer, colorectal adenoma). In some embodiments, such methods involve obtaining a stool sample from a subject (e.g., a human at risk for developing colorectal cancer; a human undergoing a routine physical examination), detecting the presence, absence, or level (e.g., mutation frequency or score, methylation frequency or score) of one or more markers specific for a colorectal neoplasm in or associated with the stool sample (e.g., specific for a colorectal neoplasm) in the stool sample, and generating a risk profile for developing colorectal neoplasm (e.g., colorectal cancer, colorectal adenoma) based upon the detected level (score, frequency) or presence or absence of the indicators of colorectal neoplasia. For example, in some embodiments, a generated risk profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers as determined with the methods of the present invention. In some embodiments, the presence and absence of specific markers as determined with the methods of the present invention are inputed into such an algorithm, and the risk profile is reported based upon a comparison of such input with established norms (e.g., established norm for pre-cancerous condition, established norm for various risk levels for developing colorectal cancer, established norm for subjects diagnosed with various stages of colorectal cancer). In some embodiments, the risk profile indicates a subject's risk for developing colorectal cancer or a subject's risk for re-developing colorectal cancer. In some embodiments, the risk profile indicates a subject to be, for example, a very low, a low, a moderate, a high, and a very high chance of developing or re-developing colorectal cancer. In some embodiments, a health care provider (e.g., an oncologist) will use such a risk profile in determining a course of treatment or intervention (e.g., colonoscopy, wait and see, referral to an oncologist, referral to a surgeon, etc.).

### EXAMPLES

The invention now being generally described, will be more readily understood by reference to the following example, which is included merely for purposes of illustration of certain aspects and embodiments of the present invention, and are not intended to limit the invention.

### Example 1

### Quantitative Stool DNA Testing for Detection of Both Colorectal Cancer and Advanced Adenoma

Subjects

Two hundred and one subjects, including 74 patients with CRC, 27 with an adenoma ≧1 cm, and 100 with normal colonoscopy, were all recruited at a major research hospital. The demographic and clinical characteristics of these subjects are shown in Table 1.

TABLE 1
Clinical characteristics of subjects.

| | Cancer | Adenoma | Normal |
|---|---|---|---|
| Number | 74 | 27 | 100 |
| Median Age (Range) | 61 yrs (40-87) | 67 yrs (50-82) | 59 yrs (28-81) |
| Sex (M/F) | 52/22 | 15/12 | 37/63 |
| Location | 29/45 | 17/10 | |
| (Proximal/Distal) | | | |
| Dukes Stage | 13/16/27/18 | | |
| (A/B/C/D) | | | |
| Grade (1/2/3/4) or | 0/4/55/5 | 20/5 | |
| Dysplasia (Low/High) | | | |

Stool Collection and DNA Extraction

Stools were collected more than 2 weeks following any colorectal diagnostic procedure or cathartic preparation and prior to either endoscopic or surgical neoplasm resection. Patients collected whole stools in a preservative buffer (0.5 mol/L Tris, 10 mmol/L NaCl, 150 mmol/L EDTA, pH 9.0), and shipped to the analysis facility within 48 hours. Once a stool arrived in the laboratory, it was weighed and homogenized. One aliquot equivalent to 10 g stool was used for stool DNA extraction, and the rest was stored at −80° C. in aliquots. Crude stool DNA was extracted with isopropanol, precipitated with ethanol, and AMA in 7.5 ml 1×TE buffer.

Real-Time Alu PCR

Human DNA in crude stool DNA was quantified using a real-time Alu PCR method. Primers specific for the human Ala sequences, sense: (SEQ ID NO: 1) 5′ ACG CCT GTA ATC CCA GCA CTT 3′; and antisense: (SEQ ID NO: 2) 5′ TCG CCC AGG CTG GAG TGC A 3′ were used to amplify sequences about 245 bp inside All repeats. Crude stool DNA was diluted 1 to 100 with nuclease-free water for PCR amplification. One μL water-diluted stool DNA was amplified in a total volume of 25 μL containing 1×iQ™ SYBR® Green Supermix (BioRad), 200 nM each primer under the following conditions: 95° C. for 3 minutes, followed by 23 cycles of 95° C. for 30 seconds, 60° C. for 30 seconds, and 72° C. for 40 seconds in a real-time iCycler (BioRad). Standard curve was created for each plate by amplifying 10-fold serially diluted human genomic DNA samples (Novagen, Madison, Wis.). A melting curve was generated after each PCR reaction to confirm that only one product was amplified for all samples. Amplification was carried out in 96-well plates in an iCycler (BioRad). Each plate consisted of stool DNA samples and multiple positive and negative controls. Each assay was performed in duplicate.

Sequence-Specific Capture

Target sequences in APC, OAS and BMP3 genes were enriched and purified from crude stool DNA using sequence-specific capture. Each capture reaction was performed by adding 600 μL of crude stool DNA to an equal volume of 6 mol/L UltraPure™ guanidine isothiocyanate solution (Invitrogen, Carlsbad, Calif.) containing a pool of biotinylated sequence-specific oligonucleotides (20 μmol total). Probe sequences for capturing APC gene were (SEQ ID NO: 3) 5′-CAGATAGCCCTGGACAAACCATGCCACCAAGCAGAAG-3′, (SEQ ID NO: 4) 5′-TTCCAGCAGTGTCACAGCACCCTAGAACCAAATCCAG-3′, and (SEQ ID NO: 5) 5′-ATGACAATGGGAATGAAACAGAATCAGAGCAGCCTAAAG-3′, and for capturing KRAS and BMP3 were (SEQ ID NO: 6) 5′-GTGGACGAATATGATCCAACAATAGAGGTAAATCTTG-3′ and (SEQ ID NO: 7) 5′-ACTTGCTGCGCTGACCCAGCGCAGCCTGACAGGTG-3′, respectively.

After one overnight incubation at room temperature, 100 μL prepared Dynabeads® M-280 streptavidin (Invitrogen) was added to the solution, and the sample was incubated for one hour at room temperature. The bead/hybrid capture complexes were that washed 2 times with 1×B+W buffer (1.0 M NaCl, 5 mM Tris-HCl [PH 7.5], 0.5 mM EDTA), and the captured DNA was eluted into 50 μL 1×TE buffer.

Digital Melt Curve (DMC) Assay

DMC assay was performed as described previously (see, e.g., Zou et al. (2009) Gastroeneterol. 136:459-470; herein incorporated by reference in its entirety). Copies of target gene sequences were initially quantified with real-time PCR. Digital PCR was performed by adding 500 gene copies in 500 μL PCR reaction mix with 2×pfx amplification buffer (Invitrogen), 0.3 mM each dNTP, 1 mM MgSO4, 0.02 unit/μL Platinum® pfx polymerase (Invitrogen), 0.1 unit/μL LcGreen⁺ dye (Idaho Tech, Salt Lake City, Utah), and 200 nM forward and reverse primers, and then dispersed into one quarter (96 wells) of one 384-well PCR plate with each'well containing 5 gene copies in 5 μL reaction volume. PCR amplification for APC gene included 95° C. 5 min, 50 cycles at 95° C. 30 sec, 64° C. 30 sec, and 72° C. 30 sec, and final denature at 95° C. 30 sec and 28° C. 30 sec to generate heteroduplex, and for KRAS gene included 95° C. 5 rain, 50 cycles at 95° C. 15 sec and 65° C. 30 sec, and final denature at 95° C. 30 sec and 28° C. 30 sec to generate heteroduplex. Two pairs of primers were used to scan APC gene mutation cluster region, included APC region 1 primers (SEQ ID NO: 8) 5′-TTCATTATCATCTTTGTCATCAGC-3′ and (SEQ ID NO: 9) 5% CGCTCCTGAAGAAAATTCAA-3′ targeting codons 1286-1346, and APC region 2 primers (SEQ ID NO: 10) 5′-CAGGAGACCCCACTCATGTT-3′ and (SEQ ID NO: 11) 5′-TGGCAAAATGTAATAAAGTATCAGC-3′ targeting codons 1394-1480, respectively. Primers used to scan KRAS mutations at codons 12 &13 were (SEQ ID NO: 12) 5% AGGCCTGCTGAAAATGACTG-3′ and (SEQ ID NO: 13) 5′-TTGTTGGATCATATTCGTCCAC-3′. Post-PCR plates were directly scanned in a LightScanner mutation analyzer (Idaho Tech) within a melt range of 75-95° C. Scanning one 384-well plate with four DMC assays required 8-10 min. Wells showing melt curve shifts resulting from the formation of mutant/wild-type heteroduplex were considered positive. Mutation score per assay was calculated based on the number of positive wells per 96 wells, i.e. mutation score=number of positive wells per plate, and mutation frequency was quantified as the ratio of mutation score to total gene copy numbers dispersed into 96 wells, i.e. mutation frequency=mutation score I number of gene copies per plate.

Bisulfite Treatment and Quantitative Methylation-Specific PCR (qMSP)

The BMP3 promoter region enriched by sequence-specific capture was treated by bisulfate using the EZ DNA Methylation Kit (Zymo Research, Orange, Calif.) and eluted in 10 μL of elution buffer. Two μL bisulfite-treated DNA was used as a template for methylation quantification with a fluorescence-based real-time PCR as described previously (see, e.g., Zou et al (2007) Cancer Epidemiol. Biomarkers 16:2686-2696; herein incorporated by reference in its entirety). Primers and probe targeting the bisulfite-modified methylated BMP3 promoter were used to quantify methylated BMP3 copies in stool DNA. PCR reactions were performed in a volume of 25 μl consisting of 600 nM of each primer; 200 of nM probe; 0.75 units of platinum Taq polymerase (Invitrogen, Carlsbad, Calif.); 200 μM each of dNTP; 16.6 mM ammonium sulfate (Sigma, St. Louis, Mo.); 67 mM Trizma (Sigma); 6.7 mM MgCl₂; 10 mM mercaptoethanol; and 0.1% DMSO.

Amplifications were performed in 96-well plates in a real-time iCycler (BioRad, Hercules, Calif.) under the following conditions: 95° C. for 2 min, followed by 50 cycles of 95° C. for 10 s and 62° C. for 60 s. A standard curve generated by 5-fold serial dilution of bisulfite-treated CpGenome™ Universal Methylated DNA (Chemicon) was used to quantify methylated gene copies. Each plate consisted of bisulfite-treated DNA samples, positive and negative controls, and water blanks.

Statistical Analysis

Wilcoxon Rank Sum test was used to compare the marker levels between each of the three different stool groups, and evaluate the association of marker levels with tumor location, gender, Dukes stage, and differentiation grade. Correlation of marker levels with tumor size and patient age was calculated with Logistic procedure. Chi-Square and Fisher exact tests were used to evaluate the association of detection rates of marker panel with clinical characteristics. Receiver Operating Curve (ROC) was constructed to compare methylation level in cancers or adenomas versus normal subjects, and area under the curve (AUC) value was also calculated for each curve. Sensitivities were calculated at 90% specificity for single markers and marker panel, and at 85% for marker panel as well. Statistical analysis was conducted with SAS software (SAS Institute, Cary, N.C.).

Results and Discussion

Single Markers.

Human DNA concentration in stool was quantified with real-time Alu PCR. Median of fecal human DNA concentration was respectively 421 (range, 0-19140), 85.6 (range, 0-6260), and 11.4 (range, 0-30000) ng/g stool for CRC patients, adenoma patients, and normal controls (p<0.001, cancer or adenoma vs. normal control). At a specificity of 90%, fecal human DNA concentration as a marker detected 65% CRCs and 41% advanced adenomas (Table 2). Corresponding cutoff was >133 ng/g stool. AUC value was 0.83 for CRC vs. normal control and 0.71 for advanced adenoma vs. normal control.

Mutation scores and frequencies of KRAS and APC genes in stool were all quantified with DMC assay, and respectively calculated based on the number of positive wells per 96 wells and the ratio of mutation score to total gene copy numbers dispersed into 96 wells. Median mutation scores of KRAS, APC region 1 (codons 1286-1346), and APC region 2 (codons 1394-1480) in stool were respectively 3 (range, 0-61), 2 (range, 0-96) and 0 (range, 0-84) for CRC patients; 1 (range, 0-50), 3 (range, 0-58), and 2 (range, 0-21) for adenoma patients; and 1 (range, 0-39), 1 (range, 0-14), and 0 (range, 0-47) for normal controls. Corresponding median mutation frequencies of KRAS and APC regions 1 & 2 were 0.6% (range, 0-12.2%), 0.4% (range, 0-19.2%), and 0% (range, 0-16.8%) for CRC patients; 0.2% (range, 0-10%), 0.6% (range, 0-11.6%), and 0.4% (range, 0-4.2%) for adenoma patients; and 0.2% (range, 0-7.8%), 0.2% (range, 0-2.8%), and 0% (range, 0-7.4%) for normal controls. Mutations scores were significantly higher in stools from patients with cancers or adenomas than normal controls for KRAS (p=, cancer vs. normal; p=, adenoma vs. normal), APC region 1 (p=, cancer vs. normal; p=, adenoma vs. normal), and APC region 2 (p=, cancer vs. normal; p=, adenoma vs. normal). At a specificity of 90%, mutations of KRAS and APC detected 38 and 34% of CRCs, and 30 and 52% of advanced adenomas, respectively. Respective mutation score (frequency) cutoffs for KRAS and APC regions 1 & 2 were 5 (1.0%), 6 (1.2%), and 4 (0.8%). AUC values of KRAS and APC mutations were 0.63 and 0.65 for CRC vs. normal control, and 0.62 and 0.81 for advanced adenoma vs. normal control.

Methylated BMP3 copies were quantified with qMSP. Median of methylated BMP3 copy was 200 (range, 0-110933), 108 (range, 0-3195), and 0 (range, 0-1800) copies/g stool for CRC patients, adenoma patients, and normal controls, respectively (p<0.001, cancer or adenoma vs. normal). At a specificity of 90% percent, fecal methylated BMP3 detected 37% CRCs and 19% adenomas. Corresponding cutoff was >683 copies/g stool. AUC value was 0.69 for CRC vs. normal control and 0.66 for advanced adenoma vs. normal control.

New Quantitative Stool DNA Testing.

At a specificity of 90%, the new quantitative stool DNA testing with a DNA marker panel including human long DNA concentration, KRAS and APC mutations, and BMP3 methylation, detected 81% CRCs and 63% advanced adenomas (Table 2). At a specificity of 85%, this full marker panel detected 91% CRCs and 78% advanced adenomas. AUC value was 0.87 for CRC vs. normal control and 0.86 for advanced adenoma vs. normal control. When using a reduced marker panel of human DNA, KRAS mutation, and BMP3 methylation, the new quantitative stool DNA testing detected 81% CRCs and 56% advanced adenomas at a specificity of 90%. AUC value was 0.86 for CRC vs. normal control and 0.80 for advanced adenoma vs. normal control. Both the full and reduced marker panels detected significantly more neoplasms than any individual markers (p<0.05).

TABLE 2
Accuracy of Colorectal Neoplasm Detection by Individual and Combined
Stool Markers. At 90% specificity, a panel with markers fecal long
DNA, KRAS, APC, and BMP3 detected 90% (9/10) adenomas >3 cm
(90%, 9/10) and 47% (8/17) adenoma ≦3 cm (p = 0.03),
and 89% (40/45) CRCs at stages III-IV and 69% (20/29) at stages
I-II (p = 0.03). Neoplasm detection rates of the full panel
were not affected by tumor location and other clinical characteristics.

| | | Sensitivity % | |
|---|---|---|---|
| | Specificity % | Cancers | Adenomas |
| Individual Markers | | | |
| Human DNA level (Alu) | 90 | 66 | 44 |
| KRAS mutation | 90 | 44 | 33 |
| APC mutation | 90 | 39 | 48 |
| BMP3 methylation | 90 | 37 | 19 |
| Combined Markers | | | |
| Alu + KRAS + BMP3 | 90 | 81 | 56 |
| Alu + KRAS + APC + BMP3 | 90 | 81 | 63 |
| Alu + KRAS + APC + BMP3 | 85 | 91 | 78 |

Association with Clinical Characteristics.

Median human DNA concentration in 45 proximal CRC stools was 112 (range, 0-3160) ng/g stool and in 29 distal CRC stools was 1006 (range, 0-19140) ng/g stool (p=0.0001); and in 29 stages I & II CRC stools was 181 (range, 0-7120) ng/g stool and in 45 stages III/IV CRC cancers was 910 (range, 0-19140) ng/g stool (p=0.001). Fecal human DNA concentration in CRC stools was not associated with other clinical characteristics, including size, differentiation, age, and gender. Fecal human DNA concentration in advanced adenoma stools was not associated with clinical characteristics.

Median KRAS mutation score was 1 (range, 0-9) in 17 proximal adenoma stools and was 8 (range, 0-51) in 10 distal adenoma stools (p=0.04). KRAS mutation score in adenoma stools was not associated with other clinical characteristics, and in CRC stools was not associated with clinical characteristics. Median mutation score of APC region 1 was 4 (range, 0-52) in proximal adenoma stools and was 1.5 (range, 0-58) in distal adenoma stools (p=0.04). APC mutation score in adenoma stools was not associated with other clinical characteristics, and in CRC stools was not associated with clinical characteristics.

Median methylated BMP3 level was 545 (range, 0-23040) copies/g stool in proximal CRC stools and 0 (range, 0-110933) copies/g stool in distal CRC stools (p=0.1). Fecal methylated BMP3 copy number in CRC stools was not associated with other clinical characteristics, and in adenoma stools was not associated with clinical characteristics.

At 90% specificity, the full panel with fecal human DNA, KRAS, APC, and BMP3 detected 90% (9/10) adenomas >3 cm (90%, 9/10) and 47% (8/17) adenoma ≦3 cm (p=0.03), and 89% (40/45) CRCs at stages III-IV and 69% (20/29) at stages I-II (p=0.03). Neoplasm detection rates of the full panel were not affected by tumor location and other clinical characteristics.

Indicators (e.g., biomarkers) of colorectal neoplasms included long DNA, KRAS and APC, and the methylated marker BMP3, each assayed using techniques that allowed high sensitivity and specificity. For example, mutations in KRAS and APC mutation cluster region were detected with a scanning approach DMC assay, so each mutation site in target gene regions was included in analysis to maximize coverage. In some embodiments, KRAS mutation was detected in 38% CRC stools and 30% adenoma, which are close to the KRAS mutation frequencies in colon tissues. Furthermore, the sensitivity of DMC assay was designed at 0.2% level, which was sufficient for identifying rare mutant gene sequences exfoliated from adenomas and early cancers. Generally, the DMC assay finds use in sensitive detection of mutations in e.g., mutation cluster regions; for example, Zou et al. reported tissue-proved mutations in 90% CRC stools and 75% adenoma stools using DMC assays (Zou et al. (2009) Gastroenterol. 136:459-470; herein incorporated by reference in its entirety). Real-time Alu PCR and qMSP also provide exquisite sensitive assay for long DNA and methylation quantification. Alu repeats represent the largest family of middle repetitive sequences in human genome. An estimated half million Alu repeats are present per haploid of human genome. In stool assays, real-time Alu PCR can detect at least 0.03 genomic copy. qMSP can detect one methylated copy in 10,000 unmethylated copies. Notably, such assays techniques were quantitative so that cutoffs were adjustable for achieving optimal marker discrimination, which are superior to qualitative assays generating end-point PCR results. Qualitative assays address either sensitivity or specificity at suboptimal levels.

Stool collection with stabilization buffer was another critical element in method, system, and kit embodiments of the present invention. Facilitating the integrity of DNA samples within stool specimens allowed the possibility of including long DNA as an informative marker and provided sufficient analyte for following mutation and methylation detection. Therefore, methods, systems, and kit embodiments of the present invention allowed detection of both CRCs and advanced adenomas at high sensitivities.

Additionally, it was shown that the DMC method can be used in a stool assay system to efficiently scan the MCR of the APC gene with sufficient sensitivity to yield high detection rates of advanced adenomas. It was also shown that tools were collected in an EDTA preservative buffer to prevent human DNA from degradation (Olson et al. (2005) Diagn. Mol. Pathol. 14:183-191; Zou et al. (2006) Cancer Epidemol. Biomarkers Prev. 15:1115-1119; each herein incorporated in its entirety) and target gene sequences were enriched from stool DNA by sequencing specific capture method with complementary probes (Ahlquist et al. (2000) Gastroeneterol. 119:1219-1227; herein incorporated by reference in its entirety), so it was possible to generate PCR amplifiable APC gene sequences in all stool samples tested. Furthermore, a highly sensitive DMC assay (Zou et al. (2009 Gastroenterol. 136:459-470; herein incorporated by reference in its entirety) was employed to scan most mutation sites in APC MCR. Innovations in both stool sample preparation and mutation detection method together led to high adenoma detection rate using system, method, and kit embodiments of the present invention.

### Example 2

### Detection of Colorectal Cancer and Advanced Adenomas by Stool Assay of Individual Markers and Selected Marker Combinations

To determine the informativeness of various single markers and combinations of markers in the detection of colorectal cancers or advanced adenomas using stool sample DNA extracts from patients, the sensitivities of several single markers were compared (DNA concentration as determined by Alu PCR; mutation frequency in KRAS as determined by digital melt curve (DMC), mutation frequency in APC as determined by DMC, vimentin methylation as determined by quantitative methlation-specific PCR (qMSP), hemoglobin concentration as determined by HemoQuant Assay) and the sensitivities several combinations of markers were compared (Alu+hemoglobin; Alu+KRAS+BMP3 methylation; Alu+KRAS+BMP3 methylation+APC; Alu+KRAS+BMP3 methylation+vimentin). Assays were conducted as described in Example 1 (Alu PCR, KRAS, APC, BMP3) or as described previously (vimentin) (Zou et al. (2007) Cancer Epidemiol. Biomarkers 16:2686-2696; herein incorporated by reference in its entirety), or according the manufacturer's instructions (HemoQuant Assay). Results are shown in Table 3.

TABLE 3
Detection of colorectal cancer (CRC) and advanced
adenoma (AdvAd) by stool assay of individual
markers and selected marker combinations.

| | | Sensitivity @ Specificities | | | |
|---|---|---|---|---|---|
| | AUC | 80% | 85% | 90% | 95% |
| CRC (74) vs Normal (100) | | | | | |
| Alu (human DNA) | 0.85 | 0.80 | 0.73 | 0.62 | 0.60 |
| Kras | 0.76 | 0.58 | 0.45 | 0.34 | 0.30 |
| APC | 0.73 | 0.51 | 0.45 | 0.36 | 0.30 |
| Vimentin | 0.86 | 0.84 | 0.80 | 0.65 | 0.45 |
| Hemoglobin⁺ | 0.84 | 0.77 | 0.65 | 0.62 | 0.43 |
| Alu + hemoglobin⁺ | 0.92 | 0.84 | 0.61 | 0.81 | 0.78 |
| Alu + Kras + bmp | 0.88 | 0.84 | 0.80 | 0.70 | 0.64 |
| Alu + Kras + bmp + apc | 0.89 | 0.59 | 0.80 | 0.68 | 0.54 |
| Alu + Kras + bmp + vimentin | 0.91 | 0.82 | 0.78 | 0.70 | 0.69 |
| Adenoma (27) vs Normal (100) | | | | | |
| Alu | 0.74 | 0.52 | 0.41 | 0.41 | 0.15 |
| Kras | 0.67 | 0.41 | 0.37 | 0.37 | 0.33 |
| APC | 0.80 | 0.53 | 0.59 | 0.56 | 0.41 |
| Vimentin | 0.70 | 0.48 | 0.44 | 0.37 | 0.26 |
| Hemoglobin⁺ | 0.61 | 0.15 | 0.15 | 0.15 | 0.04 |
| Alu + hemoglobin | 0.75 | 0.56 | 0.41 | 0.41 | 0.15 |
| Alu + Kras + bmp | 0.81 | 0.67 | 0.56 | 0.48 | 0.30 |
| Alu + Kras + bmp + apc | 0.86 | 0.81 | 0.70 | 0.56 | 0.30 |
| Alu + Kras + bmp + vimentin | 0.82 | 0.57 | 0.59 | 0.44 | 0.37 |

⁺Hemoglobin quantified by HemoQuant Assay

### Example 3

### Failure of BRAF and BAT26 to Add Sensitivity in a Multimarker Assay for Detection of Colorectal Cancer and Advanced Adenoma

During development of some embodiments of the present invention, it was found that some single markers did not promote increased sensitivity of assay methods when they were included in multimarker panels. In particular, mutation marker BRAF and microsatellite instability marker BAT26 were tested to determine whether inclusion of these markers in a multimarker assay for detection of colorectal cancer or advanced adenoma would increase the sensitivity of the assay. Inclusion of these markers did not result in a detectable improvement in sensitivity.

### Example 4

### Stool APC Mutation Assay by Digital Melt Curve Scanning for Noninvasive Detection of Colorectal Adenoma

Among all mutant genes related to colorectal cancer (CRC), the APC gene represents the most informative single gene marker for stool screening because it occurs so commonly and so early in the adenoma-to-carcinoma evolution (Kinzler et al. (1996) Cell 87:159-170; Feron et al. (1990) Cell 61:759-767; Fearnhead et al. (2001) Hum. Mol. Genet. 10:721-733; Powell et al. (1992) Nature 359:235-237; each herein incorporated by reference in its entirety). Despite its attractive features as a screening marker, the APC gene remains a technical challenge to efficiently assay the numerous potential mutational sites within its large mutator cluster region spanning roughly 800 bp (Fearnhead et al. (2001) Hum. Mol. Genet. 10:721-733; herein incorporated by reference in its entirety).

During the course of developing some embodiments of the present invention, the feasibility of a stool DMC assay to deeply scan the large mutation cluster region of the APC gene and to evaluate its sensitivity and specificity for the detection of both CRC and advanced adenomas was determined.

Materials, Methods, and Patients

Patients.

A total of 201 patients were included, including 27 with an adenoma ≧1 cm, 74 patients with CRC, and 100 with normal colonoscopy recruited at a major hospital. Their demographic and clinical characteristics are shown in Table 4.

TABLE 4
Clinical characteristics of subjects.

| | Cancer | Adenoma | Normal |
|---|---|---|---|
| Number | 74 | 27 | 95 |
| Median Age (Range) | 61 yrs (40-87) | 67 yrs (50-82) | 59 yrs (33-81) |
| Sex (M/F) | 52/22 | 15/12 | 35/60 |
| Location | 29/45 | 17/10 | |
| (Proximal/Distal) | | | |
| Dukes Stage | 13/16/27/18 | | |
| (A/B/C/D) | | | |
| Grade (1/2/3/4) or | 0/4/55/5 | 20/5 | |
| Dysplasia (Low/High) | | | |

Stool Collection and DNA Extraction.

Stools were collected more than two weeks following any colorectal diagnostic procedure or cathartic preparation and prior to either endoscopic or surgical neoplasm resection. Patients collected whole stools in a preservative buffer (0.5 mol/L Tris, 10 mmol/L NaCl, 150 mmol/L EDTA, pH 9.0) (Olson et al. (2005) Diagn. Mol. Pathol. 14:183-191; Zou et al. (2006) Cancer Epidemiol. Biomarkers Prev. 15:1115-1119; each herein incorporated by reference in its entirety, and mailed to the analyzing laboratory within 48 hours. Once a stool arrived in the laboratory, it was weighed and homogenized. One aliquot equivalent to 10 g stool was used for stool DNA extraction, and the rest was stored at −80° C. in aliquots. Crude stool DNA was extracted with isopropanol, precipitated with ethanol, and eluted in 7.5 ml 1×TE buffer. Stool DNA samples were coded and blinded for the following experiments.

Sequence-Specific Capture.

The mutation cluster region in APC gene was enriched and purified from crude stool DNA using sequence-specific capture (Ahlquist et. al. (2000) Gastroenterol. 119:1219-1227; herein incorporated by reference in its entirety). Each capture reaction was performed by adding 600 μL of crude stool DNA to an equal volume of 6 mol/L UltraPure™ guanidine isothiocyanate solution (Invitrogen, Carlsbad, Calif.) containing a pool of three biotinylated sequence-specific oligonucleotides (20 pmol total). Probe sequences for capturing APC MCR were (SEQ ID NO: 3) 5′-CAGATAGCCCTGGACAAACCATGCCACCAAGCAGAAG-3′, (SEQ ID NO: 4) 5′. TTCCAGCAGTGTCACAGCACCCTAGAACCAAATCCAG-3′, and (SEQ ID NO: 5) 5% ATGACAATGGGAATGAAACAGAATCAGAGCAGCCTAAAG-3′.

After one overnight incubation at mom temperature, 100 μL prepared Dynabeads® M-280 streptavidin (Invitrogen) was added to the solution, and incubated for one hour at room temperature. The bead/hybrid capture complexes were then washed 2 limes with 1×B+W buffer (1.0 M NaCl, 5 mM Tris-HCl [PH 7.5], 0.5 mM EDTA), and the captured DNA was eluted into 50 μL 1×TE buffer.

Digital Melt Curve (DMC) Assay and Primer Selection.

DMC assay was performed (Zou et at, (2009) Gastroenterol. 136:459-470; herein incorporated by reference in its entirety). Copies of target gene sequences were initially quantified with real-time PCR. Digital PCR was conducted by adding 500 gene copies in 500 μL PCR reaction mix with 2×pfx amplification buffer (Invitrogen), 03 mM each dNTP, 1 mM MgSO4, 0.02 unit/μL Platinum® pfx polymerase (Invitrogen), 0.1 unit/μLeGreen⁺ dye (Idaho Tech, Salt Lake City, Utah), and 200 nM forward and reverse primers, and then dispersed into one quarter (96 wells) of one 384-well PCR plate with each well containing 5 gene copies in 5 μL reaction volume. PCR amplification included 95° C. S min, 50 cycles at 95° C. 30 sec, annealing temperature 30 sec, and 72° C. 30 sec, and final denature at 95° C. 30 sec and 28° C. 30 sec to generate heteroduplex. Post-PCR plates were directly scanned in a LightScanner mutation analyzer (Idaho Tech) within a melt range of 75-95° C. Scanning can one 384-well plate with four DMC assays required 8-10 minutes. Wells showing melt curve shifts resulting from the formation of mutant/wild-type heteroduplex were considered positive. Mutation score per assay was calculated based on the number of positive wells per 96 wells, i.e. mutation score=number of positive wells per plate, and mutation frequency was quantified as the ratio of mutation score to total gene copy numbers dispersed into 96 wells, i.e. mutation frequency=mutation score/number of gene copies per plate.

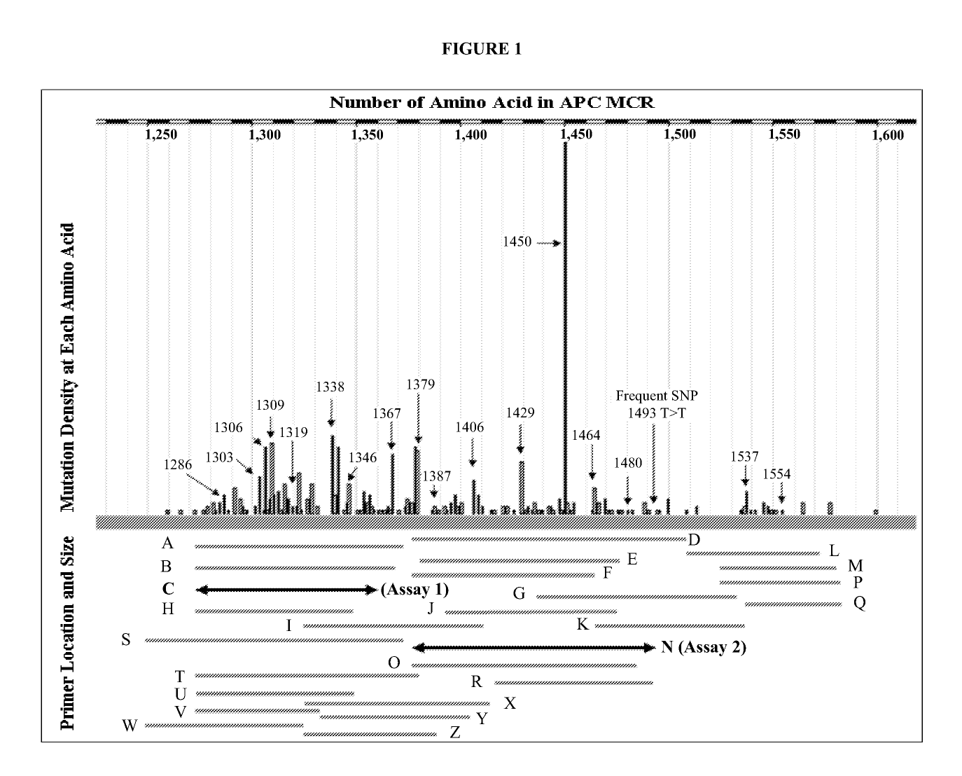

A total of 26 printer pairs were designed inside or around the APC MCR (codons 1286˜1554; FIG. 1 and Table 4) using online software Printer 3 (Whitehead Institute, MIT, USA). Primers were first tested by digitally amplifying human genomic DNA (Novagen, Madison, Wis.) with 5 genomic copies per PCR well to pick primers generating singular strong products and consistent melt curves. Four optimal primer sets that could together cover the full length of APC MCR were further tested in a subset stool samples from ten CRC patients and ten normal individuals, and two of them were excluded because they could not discriminate CRC and normal control. DMC assays, APC assays 1 and 2, designed with the final two primer sets, primer pairs C and N, were employed to deeply scan for APC mutations in 201 stool samples in blind fashion. APC assays 1 & 2 flanked codons 1286-1346 and codons 1387-1480 (Table 4), where most frequent mutation sites in APC MCR were harbored (FIG. 1). Annealing temperature was 64° C. for each assay.

TABLE 4
APC MCR scanning primers and target regions

| | | | | Common | | |
|---|---|---|---|---|---|---|
| | | | | Mutant | Product | |
| | | Primer sequence | | Codons | Size | |
| Primer Name | | (5′ to 3′) | | Targeted | (bp) | Note |
| A | Sense | (SEQ ID NO: 8) | TTCATTATCATCTTTGTCATCAGC | 1286-1367 | 300 | |
| | Antisense | (SEQ ID NO: 14) | TGTTCAGGTGGACTTTTGG | | | |
| B | Sense | (SEQ ID NO: 8) | TTCATTATCATCTTTGTCATCAGC | 1286-1353 | 280 | |
| | Antisense | (SEQ ID NO: 15) | TGTCTGAGCACCACTTTTGG | | | |
| C | Sense | (SEQ ID NO: 8) | TTCATTATCATCTTTGTCATCAGC | 1286-1346 | 250 | Assay 1 |
| | Antisense | (SEQ ID NO: 9) | CGCTCCTGAAGAAAATTCAA | | | |
| D | Sense | (SEQ ID NO: 10) | CAGGAGACCCCACTCATGTT | 1387-1489 | 391 | |
| | Antisense | (SEQ ID NO: 16) | CACTCAGGCTGGATGAACAA | | | |
| E | Sense | (SEQ ID NO: 10) | CAGGAGACCCCACTCATGTT | 1387-1463 | 293 | |
| | Antisense | (SEQ ID NO: 17) | GCAGCATTTACTGCAGCTTG | | | |
| F | Sense | (SEQ ID NO: 18) | CCCCACTCATGTTVAGCAGA | 1387-1450 | 249 | |
| | Antisense | (SEQ ID NO: 19) | TCTTTTCAGCAGTAGGTGCTTT | | | |
| G | Sense | (SEQ ID NO: 20) | CATGCCACCAAGCAGAAGTA | 1450-1489 | 233 | |
| | Antisense | (SEQ ID NO: 16) | CACTCAGGCTGGATGAACAA | | | |
| H | Sense | (SEQ ID NO: 8) | TTCATTATCATCTTTGTCATCAGC | 1286-1319 | 227 | |
| | Antisense | (SEQ ID NO: 21) | CTTTGTGCCTGGCTGATTCT | | | |
| I | Sense | (SEQ ID NO: 22) | CCCTAGAACCAAATCCAGCA | 1338-1400 | 249 | |
| | Antisense | (SEQ ID NO: 23) | CCACTGCATGGTTCACTCTG | | | |
| J | Sense | (SEQ ID NO: 24) | TTTGAGAGTCGTTCGATTGC | 1406-1463 | 239 | |
| | Antisense | (SEQ ID NO: 17) | GCAGCATTTACTGCAGCTTG | | | |
| K | Sense | (SEQ ID NO: 25) | GGACCTAAGCAAGCTGCAGTA | 1480-1513 | 208 | |
| | Antisense | (SEQ ID NO: 26) | TCCCATTGTCATTTTCCTGA | | | |
| L | Sense | (SEQ ID NO: 27) | GAGCCTCGATGAGCCATTTA | 1537-1554 | 192 | |
| | Antisense | (SEQ ID NO: 28) | TCAATATCATCATCATCTGAATCATC | | | |
| M | Sense | (SEQ ID NO: 29) | ATGCCTCCAGTTCAGGAAAA | 1537-1554 | 183 | |
| | Antisense | (SEQ ID NO: 30) | TGTTGGCATGGCAGAAATAA | | | |
| N | Sense | (SEQ ID NO: 10) | CAGGAGACCCCACTCATGTT | 1387-1480 | 346 | Assay 2 |
| | Antisense | (SEQ ID NO: 11) | TGGCAAAATGTAATAAAGTATCAGC | | | |
| O | Sense | (SEQ ID NO: 10) | CAGGAGACCCCACTCATGTT | 1387-1469 | 324 | |
| | Antisense | (SEQ ID NO: 31) | AGCATCTGGAAGAACCTGGA | | | |
| P | Sense | (SEQ ID NO: 29) | ATGCCTCCAGTTCAGGAAAA | 1537-1554 | 183 | |
| | Antisense | (SEQ ID NO: 30) | TGTTGGCATGGCAGAAATAA | | | |
| Q | Sense | (SEQ ID NO: 32) | TCAGAGCAGCCTAAAGAATCAA | 1547-1554 | 141 | |
| | Antisense | (SEQ ID NO: 30) | TGTTGGCATGGCAGAAATAA | | | |
| R | Sense | (SEQ ID NO: 33) | GGCATTATAAGCCCCAGTGA | 1463-1480 | 231 | |
| | Antisense | (SEQ ID NO: 34) | GGCAAAATGTAATAAAGTATCAGCA | | | |
| S | Sense | (SEQ ID NO: 35) | TGCCACTTGCAAAGTTTCTTC | 1286-1367 | 389 | |
| | Antisense | (SEQ ID NO: 36) | AGTGTTCAGGTGGACTTTTGG | | | |
| T | Sense | (SEQ ID NO: 8) | TTCATTATCATCTTTGTCATCAGC | 1286-1367 | 327 | |
| | Antisense | (SEQ ID NO: 37) | AACATGAGTGGGGTCTCCTG | | | |
| U | Sense | (SEQ ID NO: 8) | TTCATTATCTTCTTTGTCATCAGC | 1286-1319 | 227 | |
| | Antisense | (SEQ ID NO: 21) | CTTTGTGCCTGGCTGATTCT | | | |
| V | Sense | (SEQ ID NO: 8) | TTCATTATCATCTTTGTCATCAGC | 1286-1319 | 182 | |
| | Antisense | (SEQ ID NO: 38) | TGCTGGATTTGGTTCTAGGG | | | |
| W | Sense | (SEQ ID NO: 39) | TGCCACTTGCAAAGTTTCTT | 1286-1319 | 245 | |
| | Antisense | (SEQ ID NO: 40) | GTGACACTGCTGGAACTTCG | | | |
| X | Sense | (SEQ ID NO: 22) | CCCTAGAACCAAATCCAGCA | 1346-1400 | 253 | |
| | Antisense | (SEQ ID NO: 41) | CATTCCACTGCATGGTTCAC | | | |
| Y | Sense | (SEQ ID NO: 42) | TGCAGGGTTCTAGTTTATCTTCA | 1346-1379 | 199 | |
| | Antisense | (SEQ ID NO: 43) | CTGGCAATCGAACGACTCTC | | | |
| Z | Sense | (SEQ ID NO: 22) | CCCTAGAACCAAATCCAGCA | 1346-1379 | 179 | |
| | Antisense | (SEQ ID NO: 44) | AAGTACATCTGCTAAACATGAGTGG | | | |

Statistical Analysis.

Analysis of variance using log transformed marker levels was used to compare the marker levels between each of the three different stool groups, and evaluate the association of marker levels with tumor location, gender, Dukes stage, size, age, and differentiation grade. The combination of APC assays 1 and 2 was calculated as a sum of the two marker levels as well as weighted sum according to the coefficients from the best fitting linear discriminate equation. Chi-Square and Fisher exact tests were used to evaluate the association of detection rates of the combination of APC assays 1 & 2 with clinical characteristics. The area under the Receiver Operating Curve (ROC) was used to estimate the discriminate ability of mutation levels in cancers or adenomas versus normal subjects. Sensitivities were estimated at a corresponding 90% specificity.

Results

Design of Stool Assay.

The mutation cluster region (MCR) was targeted for assay development, as it harbors approximately 80% of somatic mutations in APC gene. There were two obstacles to overcome, including analyte enrichment and sensitive analysis of the numerous mutation sites in the APC MCR. The first one was solved by specifically capturing target sequences with biotinylated complementary probes and streptavidin-coated magnetic beads. To solve the second, DMC assays were selected to adequately scan the APC MCR using a minimal number of primer sets. Primer sets were designed within the following constraints: 1) the maximal amplicon length for accurate melt curve analysis could not exceed 400 bp, so multiple primer sets were required to optimally cover the APC MCR; 2) spans with the highest densities of mutations (i.e., between codons 1286-1379, codons 1387-1499, and codons 1537-1554 would need to be included (FIGS. 1); and 3) a common single nucleotide polymorphism (SNP) at codon 1493 (102688G>A,1493T>T) would need to be avoided (FIG. 1).

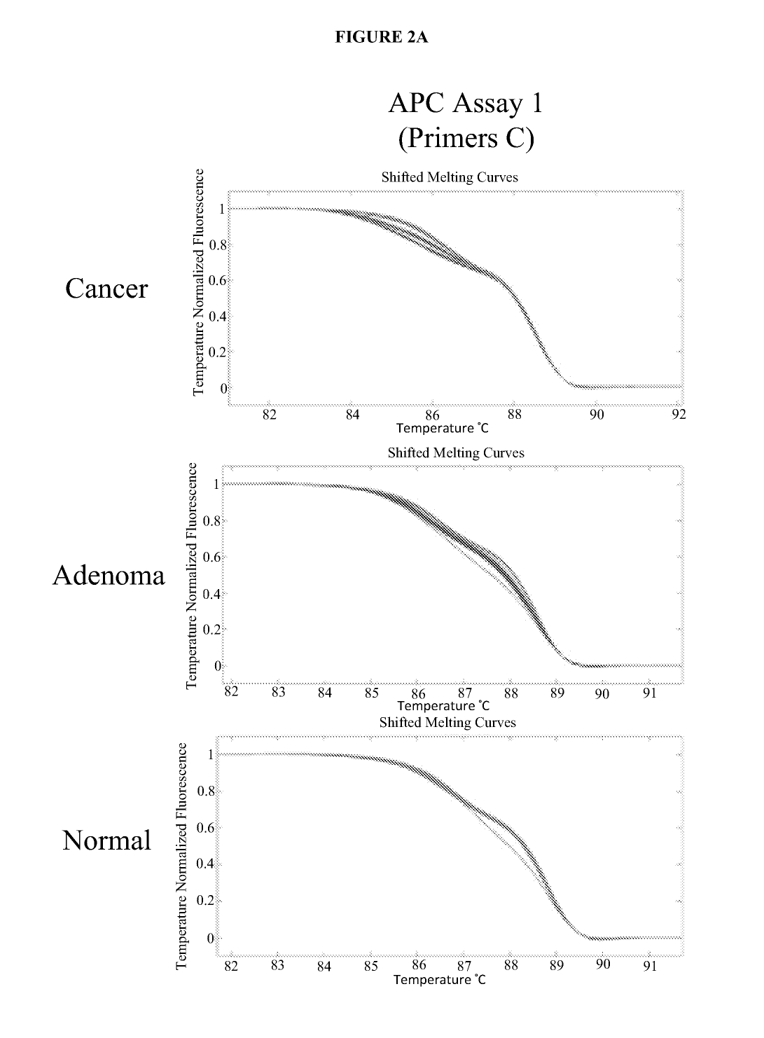

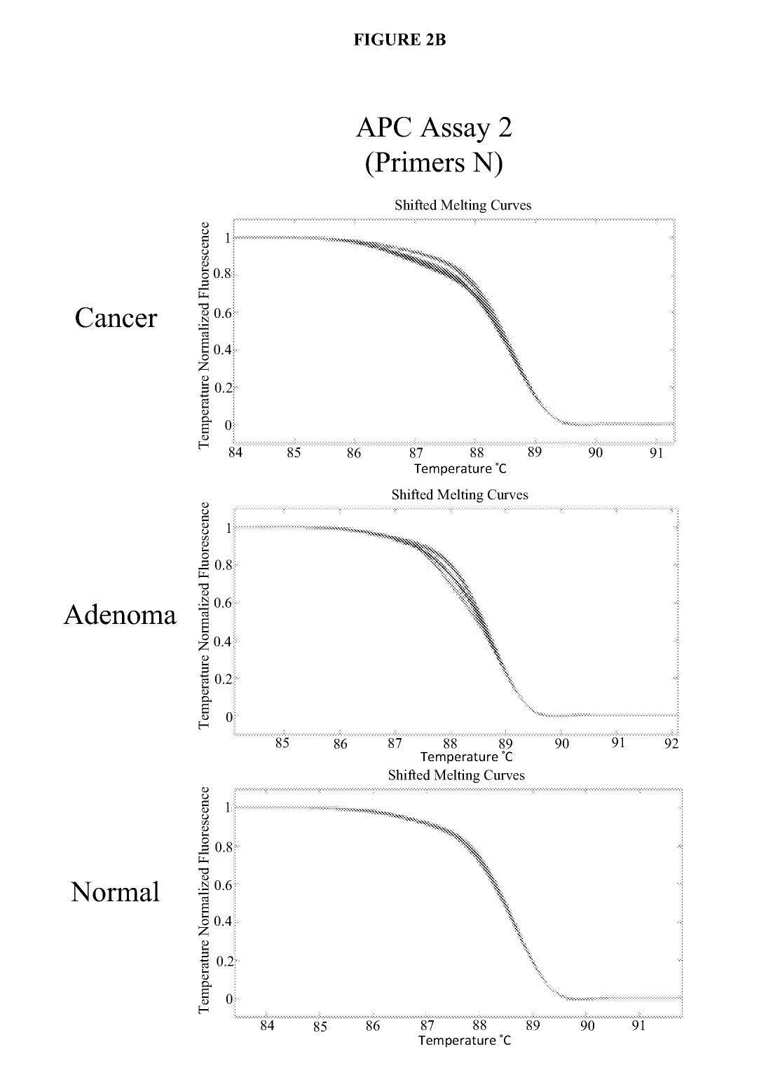

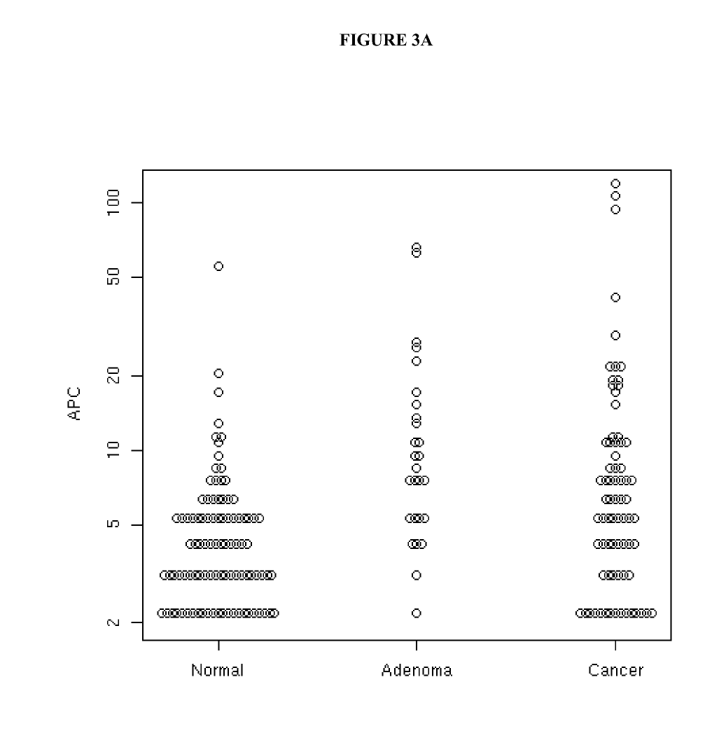

Twenty-six primers sets coded by letter and designed to flank the high-density mutation spans were tested by DMC assay. Four of them (primer pairs C, L, N, and Y) together allowed full coverage of APC MCR, minus the intentional exception of the 29 codons around the frequent SNP at 1493 (FIG. 1), and provided consistent melt curves. The target regions of primers C and N contained substantially more frequent mutation sites than those of primers L and Y (FIG. 1). DMC assays with primers C and N (designated APC assays 1 and 2 (FIG. 2)) were found to detect more APC mutations in stools from a preliminary set of 10 CRC cases than from 10 normal controls. In contrast, stool DMC assays with primers L and Y failed to discriminate CRC cases from normal controls in the preliminary sets. Thus, only APC assays 1 and 2 were employed in the clinical pilots study.

Clinical Pilot Study.

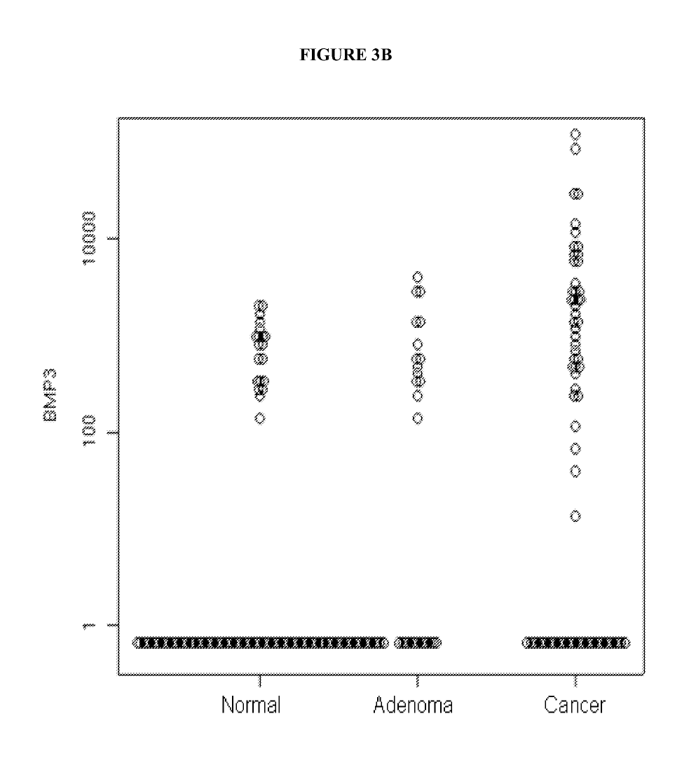

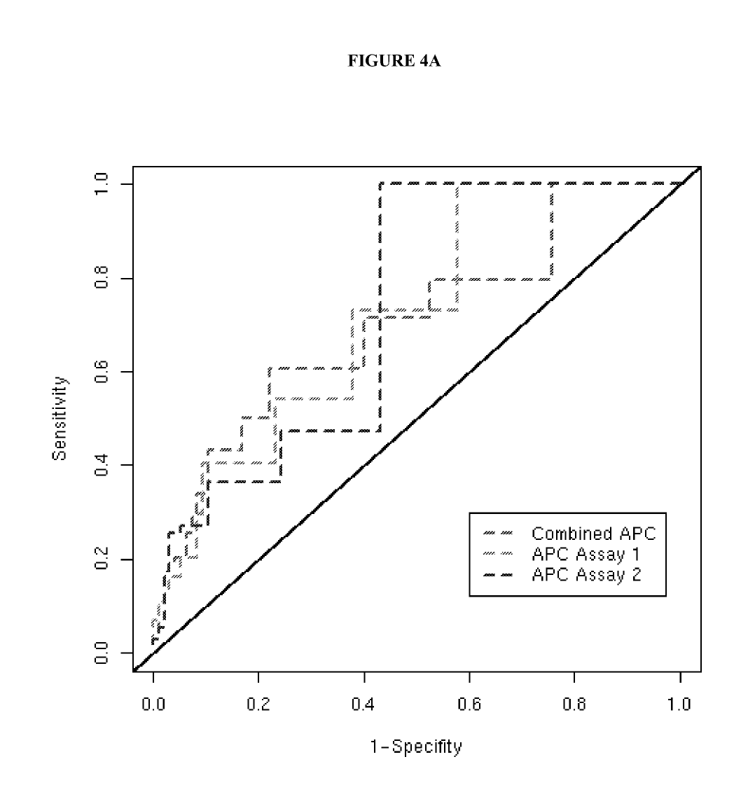

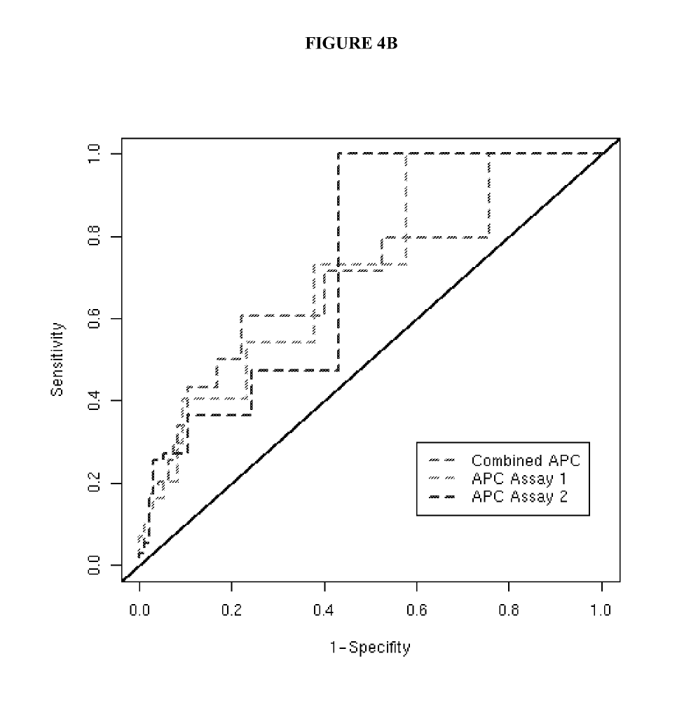

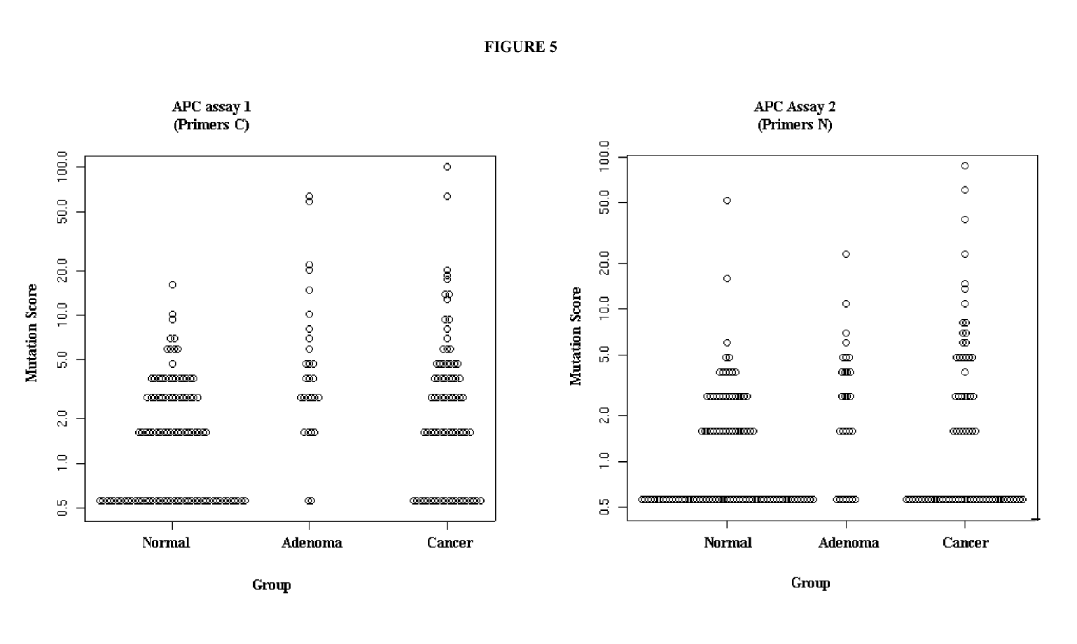

Stool APC scanning by combined assays 1 and 2 detected 52% (14/27) of advanced adenomas and 34% (25/74) of CRCs at a specificity of 90% (FIG. 2 and FIG. 3). AUC value by combined APC stool assays was 0.81 for advanced adenoma vs. normal control and 0.65 for CRC vs. normal control (FIG. 4). Median mutation scores detected by APC assays 1 and 2 were respectively 3 (range, 0-58) and 2 (range, 0-21) for adenoma patients; 2 (range, 0-96) and 0 (range, 0-84) for CRC patients; and 1 (range, 0-14) and 0 (range, 0-47) for normal controls (FIG. 5). Corresponding median mutation frequencies were 0.6% (range, 0-11.6%) and 0.4% (range, 0-4.2%) for adenoma patients; 0.4% (range, 0-19.2%) and 0% (range, 0-16.8%) for CRC patients; and 0.2% (range, 0-2.8%) and 0% (range, 0-7.4%) for normal controls. Mutation score cutoffs for APC assays 1 and 2 were respectively 6 and 4; corresponding mutation frequencies were 1.2% and 0.8%. Mutation scores were significantly higher in stools from patients with advanced adenomas than in those from normal controls for APC assay 1 (p=0.0001) and APC assay 2 (p=0.005); in cancer stools than in normal stools for APC assay 1 (p=0.0018) but not for APC assay 2 (p=0.019); in adenoma stools than in cancer stools for APC assay 1 (p=0.0115), but not for APC assay 2 (p=0.2495). Median mutation score of stool APC assay 1 was 4 (range, 0-52) with proximal adenomas and 1.5 (range, 0-58) with distal adenomas (p=0.2725). APC mutation score was not associated with other clinical characteristics.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for detecting colorectal neoplasia in a subject comprising:
a) obtaining DNA from an excreted stool sample of said subject, wherein said DNA is substantially intact relative to DNA obtained from unexcreted stool from said subject;
b) determining the level, presence, or frequency of indicators of colorectal neoplasia in said DNA from said excreted stool sample, said indicators of types selected from the group consisting of a nucleic acid polymer with altered methylation, one or more mutated nucleic acid polymers, and long DNA.

2. The method of claim 1, wherein said nucleic acid polymer with altered methylation comprises a region selected from the group consisting of a CpG island and a CpG island shore.

3. The method of claim 2, wherein said CpG island or shore is present in a coding region or a regulatory region of a gene selected from the group consisting of bmp-3, bmp-4, SFRP2, vimentin, septin9, ALX4, EYA4, TFPI2, NDRG4, and FOXE1.

4. The method of claim 2, wherein said determining of the level of altered methylation of a nucleic acid polymer comprises a determination selected from the group consisting of determining the methylation score of said CpG island or island shore and determining the methylation frequency of said CpG island or island shore.

5. (canceled)

6. The method of claim 1, wherein said determining of the level of a nucleic acid polymer with altered methylation is achieved by a technique selected from the group consisting of methylation-specific PCR, quantitative methylation-specific PCR, methylation-sensitive DNA restriction enzyme analysis, quantitative bisulfite pyrosequencing, and bisulfite genomic sequencing PCR.

7. The method of claim 1, wherein said mutated nucleic acid polymer comprises a point mutation in a gene selected from the group consisting of KRAS, APC, melanoma antigen gene, p53, and PIK3CA.

8. The method of claim 1, wherein said mutated nucleic acid polymer comprises more than one mutation in a gene selected from the group consisting of KRAS, APC, melanoma antigen gene, p53, and PIK3CA.

9. The method of claim 7, wherein said point mutation is detected by a technique selected from the group consisting of allele-specific PCR, mutant-enriched PCR, digital protein truncation test, direct sequencing, molecular beacons, and BEAMing.

10. The method of claim 8, wherein determining the level of more than one mutations comprises a determination selected from the group consisting of determining the mutation score of said nucleic acid polymer and determining the mutation frequency of said nucleic acid polymer.

11. (canceled)

12. The method of claim 10, wherein said mutation score or mutation frequency is assessed by a technique selected from the group consisting of melt curve analysis, temperature gradient gel electrophoresis, and digital melt curve assay.

13. The method of claim 1, wherein said level of long DNA is detected using Alu PCR assay.

14. The method of claim 1, further comprising: c) generating a risk profile using the results of steps a) and b).

15. The method of claim 1, wherein said colorectal neoplasm is selected from the group consisting of premalignant and malignant.

16. (canceled)

17. The method of claim 1, wherein said indicators of colorectal neoplasia comprise KRAS mutation level, APC mutation score or mutation level, BMP3 methylation level, and long DNA concentration.

18. The method of claim 1, wherein said method permits detection of colorectal cancer in said subject with a sensitivity selected from the group consisting of at least 85% at a specificity of at least 85%, at least 80% at a specificity of at least 90%, at least 75% at a specificity of at least 85%, and at least 60% at a specificity of at least 90%.

19. (canceled)

20. (canceled)

21. (canceled)

22. The method of claim 1, wherein said long DNA comprises DNA greater than 200 base pairs in length.

23. The method of claim 1, wherein detection of said one or more mutated nucleic acid polymers is selected from the group consisting of two or more mutated nucleic acid polymers are detected, three or more mutated nucleic acid polymers are detected, and four or more mutated nucleic acid polymers are detected.

24. (canceled)

25. (canceled)

26. A kit for detecting the presence of a colorectal neoplasm in a mammal, said kit comprising reagents useful, sufficient, or necessary for detecting and/or characterizing indicators of colorectal neoplasia in DNA from a stool sample, said indicators of types selected from the group consisting of a nucleic acid polymer with altered methylation, one or more mutated nucleic acid polymers, and long DNA.

27. The kit of claim 26, wherein said indicators of colorectal neoplasma are selected from the group consisting of
a) presence or frequency of mutations in coding or regulatory regions of KRAS, APC, melanoma antigen gene, p53, and PIK3CA;
b) level or frequency of CpG methylation in one or more CpG islands or CpG island shores of bmp-3, bmp-4, SFRP2, vimentin, septin9, ALX4, EYA4, TFPI2, NDRG4, FOXE1; and
c) presence or concentration of long DNA.


SEQUENCE LISTING (44 sequences)

SEQ ID NO: 1 - DNA, 21 bp, Artificial Sequence
  Synthetic
acgcctgtaatcccagcactt

SEQ ID NO: 2 - DNA, 19 bp, Artificial Sequence
  Synthetic
tcgcccaggctggagtgca

SEQ ID NO: 3 - DNA, 37 bp, Artificial Sequence
  Synthetic
cagatagccctggacaaaccatgccaccaagcagaag

SEQ ID NO: 4 - DNA, 37 bp, Artificial Sequence
  Synthetic
ttccagcagtgtcacagcaccctagaaccaaatccag

SEQ ID NO: 5 - DNA, 39 bp, Artificial Sequence
  Synthetic
atgacaatgggaatgaaacagaatcagagcagcctaaag

SEQ ID NO: 6 - DNA, 37 bp, Artificial Sequence
  Synthetic
gtggacgaatatgatccaacaatagaggtaaatcttg

SEQ ID NO: 7 - DNA, 35 bp, Artificial Sequence
  Synthetic
acttgctgcgctgacccagcgcagcctgacaggtg

SEQ ID NO: 8 - DNA, 24 bp, Artificial Sequence
  Synthetic
ttcattatcatctttgtcatcagc

SEQ ID NO: 9 - DNA, 20 bp, Artificial Sequence
  Synthetic
cgctcctgaagaaaattcaa

SEQ ID NO: 10 - DNA, 20 bp, Artificial Sequence
  Synthetic
caggagaccccactcatgtt

SEQ ID NO: 11 - DNA, 25 bp, Artificial Sequence
  Synthetic
tggcaaaatgtaataaagtatcagc

SEQ ID NO: 12 - DNA, 20 bp, Artificial Sequence
  Synthetic
aggcctgctgaaaatgactg

SEQ ID NO: 13 - DNA, 22 bp, Artificial Sequence
  Synthetic
ttgttggatcatattcgtccac

SEQ ID NO: 14 - DNA, 19 bp, Artificial Sequence
  Synthetic
tgttcaggtggacttttgg

SEQ ID NO: 15 - DNA, 20 bp, Artificial Sequence
  Synthetic
tgtctgagcaccacttttgg

SEQ ID NO: 16 - DNA, 20 bp, Artificial Sequence
  Synthetic
cactcaggctggatgaacaa

SEQ ID NO: 17 - DNA, 20 bp, Artificial Sequence
  Synthetic
gcagcatttactgcagcttg

SEQ ID NO: 18 - DNA, 20 bp, Artificial Sequence
  Synthetic
ccccactcatgtttagcaga

SEQ ID NO: 19 - DNA, 22 bp, Artificial Sequence
  Synthetic
tcttttcagcagtaggtgcttt

SEQ ID NO: 20 - DNA, 20 bp, Artificial Sequence
  Synthetic
catgccaccaagcagaagta

SEQ ID NO: 21 - DNA, 20 bp, Artificial Sequence
  Synthetic
ctttgtgcctggctgattct

SEQ ID NO: 22 - DNA, 20 bp, Artificial Sequence
  Synthetic
ccctagaaccaaatccagca

SEQ ID NO: 23 - DNA, 20 bp, Artificial Sequence
  Synthetic
ccactgcatggttcactctg

SEQ ID NO: 24 - DNA, 20 bp, Artificial Sequence
  Synthetic
tttgagagtcgttcgattgc

SEQ ID NO: 25 - DNA, 21 bp, Artificial Sequence
  Synthetic
ggacctaagcaagctgcagta

SEQ ID NO: 26 - DNA, 20 bp, Artificial Sequence
  Synthetic
tcccattgtcattttcctga

SEQ ID NO: 27 - DNA, 20 bp, Artificial Sequence
  Synthetic
gagcctcgatgagccattta

SEQ ID NO: 28 - DNA, 26 bp, Artificial Sequence
  Synthetic
tcaatatcatcatcatctgaatcatc

SEQ ID NO: 29 - DNA, 20 bp, Artificial Sequence
  Synthetic
atgcctccagttcaggaaaa

SEQ ID NO: 30 - DNA, 20 bp, Artificial Sequence
  Synthetic
tgttggcatggcagaaataa

SEQ ID NO: 31 - DNA, 20 bp, Artificial Sequence
  Synthetic
agcatctggaagaacctgga

SEQ ID NO: 32 - DNA, 22 bp, Artificial Sequence
  Synthetic
tcagagcagcctaaagaatcaa

SEQ ID NO: 33 - DNA, 20 bp, Artificial Sequence
  Synthetic
ggcattataagccccagtga

SEQ ID NO: 34 - DNA, 25 bp, Artificial Sequence
  Synthetic
ggcaaaatgtaataaagtatcagca

SEQ ID NO: 35 - DNA, 21 bp, Artificial Sequence
  Synthetic
tgccacttgcaaagtttcttc

SEQ ID NO: 36 - DNA, 21 bp, Artificial Sequence
  Synthetic
agtgttcaggtggacttttgg

SEQ ID NO: 37 - DNA, 20 bp, Artificial Sequence
  Synthetic
aacatgagtggggtctcctg

SEQ ID NO: 38 - DNA, 20 bp, Artificial Sequence
  Synthetic
tgctggatttggttctaggg

SEQ ID NO: 39 - DNA, 20 bp, Artificial Sequence
  Synthetic
tgccacttgcaaagtttctt

SEQ ID NO: 40 - DNA, 20 bp, Artificial Sequence
  Synthetic
gtgacactgctggaacttcg

SEQ ID NO: 41 - DNA, 20 bp, Artificial Sequence
  Synthetic
cattccactgcatggttcac

SEQ ID NO: 42 - DNA, 23 bp, Artificial Sequence
  Synthetic
tgcagggttctagtttatcttca

SEQ ID NO: 43 - DNA, 20 bp, Artificial Sequence
  Synthetic
ctggcaatcgaacgactctc

SEQ ID NO: 44 - DNA, 25 bp, Artificial Sequence
  Synthetic
aagtacatctgctaaacatgagtgg